(19) United States
(12) Patent Application Publication
(10) Pub. No.: US 2025/0044294 A1
(43) Pub. Date: Feb. 6, 2025

(54) COMPOSITION AND METHOD OF TREATING A CANCER THROUGH AFFECTING MEMBRANE RECEPTORS OF CANCER CELLS AND THEIR DERIVED EXTRACELLULAR VESICLES

(71) Applicant: National Yang Ming Chiao Tung University, Taipei City (TW)

(72) Inventors: Chi-Ying HUANG, Taipei City (TW); Wei-Ni TSAI, Taipei City (TW); Cayla SOLOMON, Taipei City (TW); Tai-Shan CHENG, Taipei City (TW); Ming-Hsi CHUANG, Zhubei City (TW); Ly James LEE, Taipei City (TW); Peter Mu-Hsin CHANG, Taipei City (TW); Yu-Tang HUANG, Taipei City (TW); Thi Tuong Linh NGUYEN, Taipei City (TW); Yi-Ning LO, Taipei City (TW)

(73) Assignee: National Yang Ming Chiao Tung University, Taipei City (TW)

(21) Appl. No.: 18/790,668

(22) Filed: Jul. 31, 2024

Related U.S. Application Data
(60) Provisional application No. 63/516,722, filed on Jul. 31, 2023.

(51) Int. Cl. G01N 33/574; A61K 31/4178

(52) U.S. Cl. CPC G01N 33/57484; A61K 31/4178; G01N 33/57423; G01N 33/57438; G01N 2800/52; G01N 2800/7028

(57) ABSTRACT

The present invention is related to a use of prochlorperazine (PCP), or analog thereof for treating a cancer in a subject by influencing membrane proteins and receptors and inducing alterations in the expressions of the surface marker on cancer cells and their derived extracellular vesicles. The invention method offers a novel approach for the treatment and diagnosis of cancer and metastasis. Specific surface markers serve as a potential candidate for cancer-associated extracellular vesicles (EVs) and have applications in diagnosis, prognosis, and therapeutic targeting.

### CROSS REFERENCE

This application claims priority to U.S. Provisional Patent Appl. No. 63/516,722 filed on Jul. 31, 2023, the contents of which is incorporated by reference in its entirety.

### FILED OF THE INVENTION

The invention relates to a composition and method for treating cancer by affecting membrane proteins and receptors, which leads to alterations in surface markers on cancer cells and their derived extracellular vesicles.

### BACKGROUND

Surface markers in cancer refer to proteins or molecules that are present on the surface of cancer cells. These markers can be specific to certain types of cancer or more broadly expressed across multiple cancer types. Surface markers play a crucial role in the identification, diagnosis, and targeted treatment of cancer.

Surface markers on extracellular vesicles (EVs) have gained significant interest as potential drug targets in various diseases, including cancer. EVs are small membrane-bound vesicles released by cells into the extracellular space, and they play a crucial role in intercellular communication by transferring bioactive molecules, such as proteins, nucleic acids, and lipids, between cells.

The ERBB family, also known as the HER family, consists of a group of receptor tyrosine kinases that play a crucial role in cell growth, survival, and proliferation. The family includes four members: HER1 (EGFR, ERBB1), HER2 (ERBB2), HER3 (ERBB3), and HER4 (ERBB4). High expression levels of the ERBB family are commonly associated with advanced disease and poorer prognosis in several cancer types, including pancreatic cancer, head and neck cancer, and lung cancer. Several EGFR inhibitors have been investigated as potential treatments in combination with chemotherapy agents for these cancers. However, the dysregulation of the ERBB family, including the oncogenic dimerization of EGFR with other ERBB family members, leads to the acquisition of cancer stemness properties and drug resistance. Therefore, the identification of resistance mechanisms to chemotherapy or anti-EGFR agents has prompted researchers to propose new combinatorial strategies to overcome drug resistance.

Integrins are a family of transmembrane proteins that mediate cell-cell and cell-extracellular matrix interactions. Both integrin α6 and integrin β4 are highly expressed in pancreatic cancer, correlating with more aggressive behavior and poor prognosis. They form the integrin α6 (ITGA6)/integrin β4 (ITGB4) receptor, which binds to the extracellular matrix (ECM) ligand laminin to promote cell adhesion, migration, invasion, and metastasis. These integrins contribute to the invasive nature of pancreatic cancer cells, particularly in a tumor microenvironment enriched with fibrotic stroma.

CD9 proteins are members of the tetraspanin protein family and are involved in various physiological processes, including cell motility, adhesion, and invasion. Previous studies have indicated that CD9 identifies cancer stem cells capable of reinitiating tumor formation and recapitulating the cellular heterogeneity of primary PDAC. Furthermore, CD9 mediates stromal cell signaling that promotes PDAC progression [1, 2].

Phenothiazine derivatives are a group of old drugs that have recently been studied for their potent anticancer effects. Prochlorperazine (PCP), primarily used as an antiemetic, has fewer central nervous system effects. Therefore, it is desirable to develop a new method for treating cancer by affecting membrane receptors to induce alterations in surface markers, including the ERBB family and integrins, on cancer cells and their derived extracellular vesicles. Furthermore, surface markers expressed on EVs could serve as biomarkers for selecting patients, thereby guiding prochlorperazine treatment to achieve the goal of precision medicine.

### SUMMARY OF THE INVENTION

It is unexpectedly discovered that prochlorperazine (PCP) has anti-tumor and anti-metastatic efficacy, particularly in treatment of a cancer.

In one aspect, the present invention provides a method for treating a cancer with high expression of a specific marker in a subject, comprising the steps of
(a) collecting cancer cell samples of the subject;
(b) determining whether the specific marker is overexpressed on cancer cells or their extracellular vesicles (EVs); and
(c) administering to the subject a therapeutically effective amount of prochlorperazine (PCP), or analog thereof, in combination of an administration of a chemotherapeutic drug if the specific marker is overexpressed on cancer cells or their extracellular vesicles (EVs) in the subject;
   wherein the specific marker is selected from the group consisting of ERBB family member, CD9, and integrins.

In another aspect, the present invention provides a use of prochlorperazine (PCP), or analog thereof for manufacturing a medicament for treating a cancer with high expression of a specific marker on cancer cells or their extracellular vesicles (EVs) in a subject, in combination of an administration of a chemotherapeutic drug; wherein the specific marker is selected from the group consisting of ERBB family member, CD9, and integrins.

In a further aspect, the present invention provides a pharmaceutical composition for treating a cancer with high expression of a specific marker on cancer cells or their extracellular vesicles (EVs) in a subject, comprising prochlorperazine (PCP), or analog thereof in combination of an administration of a chemotherapeutic drug; wherein the specific marker is selected from the group consisting of ERBB family member, CD9, and integrins.

In a further aspect, the present invention provides an in vitro method for diagnosing pancreatic adenocarcinoma (PDAC) in a subject comprising the steps of
(a) collecting cancer cells or extracellular vesicles (EVs) of the subject;
(b) detecting the expression of a specific surface marker on the cancer cells or EVs collected in step (a); and
(c) diagnosing the subject as suffering from PDAC if the specific surface marked is expressed in step (b);
   wherein the specific surface marker is selected from the group consisting of EGFR, Glypican 1 (GPC1), EpCAM, CD9, integrins, α-Enolase (ENO1), HER2, HER3, MET and combination thereof.

In a further aspect, the present invention provides an in vitro method for diagnosing head and neck squamous cell carcinoma (HNSCC) in a subject comprising the steps of
(a) collecting cancer cells or extracellular vesicles (EVs) of the subject;
(b) detecting the expression of a specific surface marker on the cancer cells or EVs collected in step (a), and
(c) diagnosing the subject as suffering from HNSCC if the specific surface marked is expressed in step (b),
   wherein the specific surface marker is selected from the group consisting of EGFR, HER2, HER3, MET, and combination thereof.

In a yet aspect, the present invention provides a in vitro method for diagnosing non-small-cell lung carcinoma (NSCLC) in a subject comprising the steps of
(a) collecting cancer cells or extracellular vesicles (EVs) of the subject;
(b) detecting the expression of a specific surface marker on the cancer cells or EVs collected in step (a); and
(c) diagnosing the subject as suffering from NSCLC if the specific surface marked is expressed in step (b);
   wherein the specific surface marker is selected from the group consisting of EGFR, HER3, and combination thereof.

In an embodiment of the present invention, the ERBB family member is selected from the group consisting of HER1 (epidermal growth factor receptor (EGFR)), HER2, and HER3.

In a further embodiment, the integrins is selected from the group consisting of integrin α6 subunit and integrin β4 subunit.

In one embodiment of the present invention, the specific marker is selected from the group consisting of EGFR, p-EGFR, Glypican 1 (GPC1), EpCAM, CD9, integrins, α-Enolase (ENO1), HER2, HER3, and MET.

In one embodiment of the invention, the cancer is a chemotherapeutic drug-resistant cancer.

In another embodiment of the present invention, the cancer is a metastatic cancer.

In one embodiment of the present invention, the chemotherapeutic drug is an epidermal growth factor receptor (EGFR)-tyrosine kinase inhibitor, such as afatinib.

In another embodiment of the present invention, the chemotherapeutic drug is gemcitabine.

In some embodiments of the present invention, the analog of PCP is selected from the group consisting of trifluoperazine, fluphenazine, chlorpromazine, thioridazine, and perphenazine.

In one particular example of the present invention, the analog of PCP is thioridazine.

In one embodiment of the present invention, the cancer is pancreatic cancer, head and neck cancer, or lung cancer.

In one particular example of the present invention, the pancreatic cancer is a pancreatic adenocarcinoma (PDAC).

In one particular example of the present invention, the head and neck cancer is head and neck squamous cell carcinoma (HNSCC).

In one particular example of the present invention, the lung cancer is non-small-cell lung carcinoma (NSCLC).

In a yet aspect, the present invention provides a method for treating a cancer in a subject, which comprises administering to the subject a therapeutically effective amount of PCP or analog thereof; wherein the cancer is selected from the group consisting of pancreatic cancer, head and neck cancer, and lung cancer.

In one embodiment of the invention, the method can be in combination of an administration of a chemotherapeutic drug.

According to the invention, prochlorperazine (PCP), or an analog thereof is effective in suppressing the migratory and invasive abilities of cancer cells.

According to the invention, prochlorperazine (PCP), or analog thereof is effective in suppressing pro-migratory ability of cancer cell-derived EVs.

It is to be understood that both the foregoing general description and the following detailed description are exemplary and explanatory only and are not restrictive of the invention.

### BRIEF DISCRIPTION OF THE DRAWINGS

The foregoing description and the following detailed description of the invention will be better understood when reading in conjunction with the accompanying drawings. For the purpose of illustrating the present invention, currently preferred embodiments are shown in the drawings.

FIG. 1 illustrates the two sequential treatment including pre-treatment with PCP before EGF stimulation to mimic the effect of antipsychotic treatment, and post-treatment with PCP after EGF stimulation to mimic tumor growth with drug treatment.

FIGS. 2A-2D illustrates that two sequential treatments had distinct effects on EGFR downstream signaling pathways. Western blot analysis demonstrated that pre-treatment with PCP effectively inhibited the activation of EGFR downstream proteins. Conversely, post-treatment with PCP did not impede EGFR activation but did inhibit AKT activation in SAS and HSC3 cells. FIGS. 2A and 2B show that HNSCC cells, including SAS and HSC3 were pre-treated with PCP 15 UM for 30 minutes, followed by EGF (10 ng/ml) stimulation for 15 minutes. FIGS. 2C and 2D show that SAS and HSC3 were post-treated with PCP 15 μM for 30 minutes after EGF (10 ng/ml) stimulation for 15 minutes. *p<0.05; **p<0.01; ***p<0.001, compared to control.

FIG. 3 illustrates the effect of PCP pre-treatment on reducing EGFR downstream protein expression levels in HNSCC cells; wherein HNSCC cells, including HSC3, SAS, and FaDu cells, were treated with afatinib (10 nM) and PCP (5, 10, 15 μM) for 30 minutes, followed by stimulation with EGF (10 ng/ml) for 15 minutes. Protein expression levels were then determined by western blotting. The results showed that PCP suppressed EGFR downstream protein expression levels. *p<0.05; **p<0.01; ***p<0.001, compared to control.

FIGS. 4A-4B illustrates the effect of PCP pre-treatment on inhibiting PI3K/AKT/mTOR pathway. FIG. 4A shows that HSC3 cells were pre-treated with PCP (15 μM) for 30 minutes, followed by stimulation with EGF (10 ng/ml) for 15 minutes. Protein expression levels were then determined by western blotting. FIG. 4B shows that PCP effectively suppressed PI3K/AKT downstream protein expression levels. *p<0.05; ***p<0.001, using one-way ANOVA.

FIGS. 5A-5B illustrates the effect of PCP pre-treatment and post-treatment on inhibiting AKT activation in NSCLC cells. FIG. 5A shows that A549 cells were pre-treated with PCP (15 μM) for 30 minutes, followed by stimulation with EGF (10 ng/mL) for 15 minutes. Protein expression levels were then determined by western blotting. FIG. 5B shows that A549 cells were post-treated with PCP (15 μM) for 30 minutes after EGF (10 ng/mL) stimulation for 15 minutes. Protein expression levels were then determined by western blotting. The results demonstrated that both of PCP pre-treatment and post-treatment effectively suppressed AKT activation. **p<0.01, using one-way ANOVA.

FIG. 6 illustrates that treatment with PCP (20 μM) for 6 hours suppressed AKT activation in PDAC cells.

FIGS. 7A-7B illustrates that thioridazine (THD) pre-treatment could suppress EGFR downstream activation in HNSCC cells: FIG. 7A shows that HSC3 cells were pre-treated with THD (5, 10, 15 UM) for 30 minutes, followed by stimulation with EGF (10 ng/mL) for 15 minutes. Protein expression levels were then determined by western blotting. FIG. 7B shows that pre-treatment with THD (10, 15 μM) for 30 minutes after EGF (10 ng/mL) stimulation could suppress the expressions of p-EGFR/EGFR, p-AKT/AKT, and p-ERK/ERK in HSC3 cells.

FIGS. 8A-8B illustrates that pre-treatment with PCP could suppress EGFR-p-EGFR protein-protein interactions in HNSCC cells: HSC3 cells (FIG. 8A) and SAS cells (FIG. 8B) were pre-treated with PCP (15 μM) for 30 minutes, followed by stimulation with EGF (10 ng/ml) for 15 minutes. Protein-protein interactions were then determined by the proximity ligation assay. Pre-treatment with PCP (15 μM) before EGF (10 ng/mL) stimulation could suppress EGFR-p-EGFR protein-protein interactions in both HSC3 and SAS cells. ***p<0.001, using one-way ANOVA.

FIGS. 9A-9B illustrates that pre-treatment with PCP could suppress EGFR-HER2 protein-protein interactions in HNSCC cells: HSC3 (FIG. 9A) and SAS cells (FIG. 9B) were pre-treated with PCP (15 μM) for 30 minutes, followed by stimulation with EGF (10 ng/mL) for 15 minutes. Protein-protein interactions were then determined by the proximity ligation assay. Pre-treatment with PCP (15 μM) before EGF (10 ng/mL) stimulation could suppress EGFR-HER2 protein-protein interactions in both HSC3 and SAS cells. *p<0.05; **p<0.01; ***p<0.001, using one-way ANOVA.

FIGS. 10A-10B illustrates that pre-treatment with PCP could suppress EGFR-HER3 protein-protein interactions in HNSCC cells: HSC3 (FIG. 10A) and SAS cells (FIG. 10B) were pre-treated with PCP (15 μM) for 30 minutes, followed by stimulation with EGF (10 ng/ml) for 15 minutes. Protein-protein interactions were then determined by the proximity ligation assay. Pre-treatment with PCP (15 μM) before EGF (10 ng/mL) stimulation could suppress EGFR-HER3 protein-protein interactions in both HSC3 and SAS cells. *p<0.05; **p<0.01; ***p<0.001, using one-way ANOVA.

FIG. 11 illustrates that pre-treatment with PCP could suppress EGFR-MET protein-protein interactions in HNSCC cells; wherein HNSCC cells, such as HSC3, were pre-treated with PCP (15 μM) for 30 minutes, followed by stimulation with EGF (10 ng/mL) for 15 minutes. Protein-protein interactions were then determined by the proximity ligation assay. Pre-treatment with PCP (15 μM) before EGF (10 ng/mL) stimulation could suppress EGFR-MET protein-protein interactions in HSC3 cells. ***p<0.001, using one-way ANOVA.

FIGS. 12A-12B illustrates that pre-treatment with PCP could suppress EGFR-HER3 protein-protein interactions in NSCLC cells: A549 cells (FIG. 12A) and H441 cells (FIG. 12B) were pre-treated with PCP (15 μM) for 30 minutes, followed by stimulation with EGF (10 ng/mL) for 15 minutes. Protein-protein interactions were then determined by the proximity ligation assay. Pre-treatment with PCP (15 μM) before EGF (10 ng/mL) stimulation could suppress EGFR-HER3 protein-protein interactions in both A549 and H441 cells. **p<0.01, using one-way ANOVA.

FIGS. 13A-13B illustrates that pre-treatment with PCP could suppress EGFR-p-EGFR (FIG. 13A) and EGFR-HER3 (FIG. 13B) protein-protein interactions in PDAC cells; BxPC-3 cells were pre-treated with PCP (15 μM) for 30 minutes, followed by stimulation with EGF (10 ng/mL) for 15 minutes. Protein-protein interactions were then determined by the proximity ligation assay. Pre-treatment with PCP (15 μM) before EGF (10 ng/mL) stimulation could suppress both of EGFR-p-EGFR and EGFR-HER3 protein-protein interactions in BxPC-3 cells.

FIGS. 14A-14C illustrates that combined treatment with PCP and afatinib inhibited EGFR-HER3 protein-protein interactions to overcome afatinib-induced drug resistance. FIG. 14A shows that HSC3 cells were pre-treated with PCP (15 μM) and afatinib (100 nM) for 30 minutes, followed by stimulation with EGF (10 ng/ml) for 15 minutes. Protein-protein interactions were then determined by the proximity ligation assay. FIG. 14B shows that HSC3 cells were post-treated with PCP (15 μM) and afatinib (100 nM) for 30 minutes after EGF (10 ng/ml) stimulation for 15 minutes. Protein-protein interactions were then determined by the proximity ligation assay. Both pre-treatment and post-treatment could suppress EGFR-HER3 protein-protein interactions in HSC3 cells. FIG. 14C shows that the combined treatment with PCP (15 μM) and afatinib (100 nM) for 30 minutes after EGF (10 ng/ml) stimulation could suppress the expressions of p-HER3/HER3 and p-AKT/AKT in HSC3 cells. *p<0.05; **p<0.01; ***p<0.001, using one-way ANOVA.

FIGS. 15A-15E illustrates that the combination of gemcitabine and PCP treatment exhibited significant anti-PDAC effects in pancreatic cancer patient-derived cells and their cultured tumoroids. FIG. 15A shows that 72-hour PCP treatment inhibited the expression levels of integrin α6/β4, CD9, and ERBB family members, including EGFR, HER2, and HER3, in 3R-4 tumoroids, as determined by western blotting. However, the unchanged expression levels of GPC1 highlighted the utility of the GPC1 antibody for the selection of PDAC patients. FIG. 15B shows that 72-hour PCP treatment also inhibited the expression levels of CD9 and integrin β4 in 3R-4 and 3R-5-1 tumoroids, as determined by immunofluorescence analysis. FIG. 15C shows that the combination of gemcitabine and PCP treatment exhibited significant cytotoxic effects in 3R-4 tumoroids, as demonstrated by the Cyto3D® Live-Dead Assay Kit and image analysis. FIGS. 15D-15E show that the combination of gemcitabine and PCP treatment significantly downregulated stemness and the EMT signature in 3R-4 tumoroids, as assessed by immunofluorescence analysis of the cancer stem cell marker CD44 and the EMT marker vimentin in drug-treated tumoroids. *p<0.05, **p<0.01, ***p<0.001, ****p<0.0001, compared to control.

FIG. 16 illustrates that PCP treatment significantly suppressed the migration ability of PDAC cells compared to gemcitabine treatment. The cell viability of PDAC cells, including BxPC-3, PANC-1, and AsPC-1 cells, were evaluated in both the gemcitabine and PCP treatment groups. **p<0.01, ****p<0.0001, using one-way ANOVA.

FIGS. 17A-17C illustrates that PCP treatment inhibited the functional activity of integrin α6/B4 on PDAC-derived EVs to suppress their migratory capability in treated normal cells. FIG. 17A shows that as compared with PANC-1-derived secretomes, the human pancreatic duct epithelial cells (H6C7 cells) significantly altered migratory behavior after co-culturing with BxPC-3-derived secretomes and the effect could be significantly suppressed by the treatment of PCP. FIG. 17B shows that the human lung fibroblasts (WI-38 cells) significantly altered migratory behavior after co-culturing with PDAC-derived secretomes from BxPC-3, AsPC-1, and PANC-1 cells. However, the effects of PCP treatment varied among the secretomes derived from the three PDAC cell lines. PCP treatment showed the strongest inhibition of migratory capability in BxPC-3-derived secretomes, followed by a comparatively weaker effect in AsPC-1-derived secretomes, and the least effect in PANC-1-derived secretomes. This observation was consistent with the expression levels of integrin α6/B4 among these PDAC cells. FIG. 17C shows that as compared to the vehicle (no laminin coating) group, BxPC-3-derived EVs significantly increased the migratory capability of treated H6C7 cells in the laminin coating group. Furthermore, PCP treatment significantly inhibited the functional activity of integrin α6/B4 on BxPC-3-derived EVs. *p<0.05; **p<0.01; ***p<0.001, using one-way ANOVA.

FIGS. 18A-18B illustrates that PCP treatment suppressed the migratory capability induced by NSCLC-derived EVs in treated normal cells and KRAS wild-type NSCLC cells. FIG. 18A shows that lung epithelial cells (BEAS-2B) and KRAS wild-type NSCLC cells (CL141) exhibited altered migratory behavior after co-culturing with NSCLC-derived secretomes from H441 cells. PCP treatment demonstrated an inhibitory effect on the migratory capability induced by NSCLC-derived secretomes. FIG. 18B shows that NSCLC-derived EVs significantly increased the migratory capability of treated cells; however, PCP treatment could downregulate this increased migratory capability in treated cells. **p<0.01; ***p<0.001, using one-way ANOVA.

FIGS. 19A-19D illustrates the validation of co-expressions of membrane proteins on cancer-associated EVs using TIRF technology. FIG. 19A shows that the identification of membrane proteins on tumor-associated EVs was established using TIRF on the biochip. FIG. 19B shows that the combinations of surface markers such as CD9 and ITGA6 demonstrated potential for distinguishing stage IV patients from early-stage PDAC patients. Additionally, combinations including EGFR, GPC1, EpCAM, and ITGA6 showed potential as biomarkers for the detection of early-stage PDAC. FIG. 19C shows that the combinations including CD9, and ITGA6 demonstrated high potential as candidate PDAC-associated markers. The expression levels of CD9 and ITGA6 on EVs from PDAC cells, including AsPC-1, BxPC-3, and PANC-1, were quantified with the total fluorescence intensity. FIG. 19D shows that PDAC cells, such as BxPC-3, expressing higher levels of CD9 and ITGA6 exhibited greater sensitivity to PCP treatment. **p<0.01; ***p<0.001, using one-way ANOVA.

FIGS. 20A-20C illustrates the protein expression levels demonstrated by western blots. FIG. 20A illustrates the validation of the surface markers for PDAC-derived extracellular vesicles: integrin β4, integrin α6, GPC1, and ENO1. FIG. 20B illustrates the validation of the surface markers for PDAC-derived extracellular vesicles: EGFR, p-EGFR, and EpCAM. FIG. 20C illustrates the validation of the surface markers for PDAC-derived extracellular vesicles: HER2, and MET were all expressed on PDAC-derived EVs. Compared to MSC-derived EVs, integrin β4, integrin α6, GPC1, ENO1, HER2, and MET showed the potential to be surface markers for candidate PDAC-associated EVs.

FIGS. 21A-21B illustrates that the binding of the ECM ligand laminin activated the cancer cell proliferation and increased the drug sensitivity to PCP treatment in BxPC-3 cells. FIG. 21A shows that after laminin binding to integrin α6/β4, PANC-1 cells exhibited increased expression of FAK/AKT downstream proteins. FIG. 21B shows that BxPC-3 cells displayed a significantly increased cell proliferation rate following 48 hours of laminin binding. Moreover, BxPC-3 cells showed a significantly higher increase in drug sensitivity to PCP treatment compared to the vehicle group after 48 hours of treatment. #p<0.05; **p<0.01; ***p<0.001, compared to control/vehicle.

FIGS. 22A-22B illustrates that PCP treatment inhibited integrin α6/β4 protein expression on EVs derived from BxPC-3 (FIG. 22A) and PANC-1 cells (FIG. 22B). BxPC-3 cells, which express elevated levels of integrin α6/β4, exhibited higher sensitivity to PCP compared to PANC-1 cells. *p<0.05; **p<0.01, compared to control.

FIGS. 23A-23C illustrates that PCP exhibited strong anti-metastatic effects by modulating the secretion of ITGB4+CD9+ EVs and downregulating the co-expression levels of CD9 and integrin β4 on EVs from PDAC cells. FIG. 23A shows that PCP significantly downregulated the secretion of CD9-EVs while upregulating the secretion of CD63-EVs from BxPC-3 cells. FIG. 23B shows that the effect of CD9/CD63-blocking antibodies, utilized with CD9/CD63-magnetic beads, on PANC-1 cell migration induced by BxPC-3-derived secretomes was evaluated using a transwell migration assay. PANC-1 cells significantly enhanced migratory capacity when co-cultured with the secretomes from BxPC-3 cells for 20 hours. Blocking CD9 proteins on BxPC-3-derived secretomes significantly reduced the migratory ability of PANC-1 cells, indicating a specific pro-migratory role of BxPC-3-derived CD9-EVs. FIG. 23C shows that PCP treatment significantly suppressed the secretion of ITGB4+CD9+ EVs and downregulated the co-expression levels of CD9 and integrin β4 proteins on ITGB4+CD9+ EVs from BxPC-3 cells. Quantification of EV populations and assessment of protein co-expression levels were performed using NanoFCM analysis. *p<0.05; **p<0.01, compared to control.

FIG. 24 illustrates that the combination of gemcitabine and PCP treatment exhibited a synergistic cytotoxic effect in pancreatic cancer patient-derived cells and their cultured tumoroids (SP-2). **p<0.01, ***p<0.001, ****p<0.0001, compared to the PCP treatment alone.

FIG. 25 illustrates several specific surface marker combinations expressed in different cancer types. ‘ND’ indicates that the expression levels were not determined.

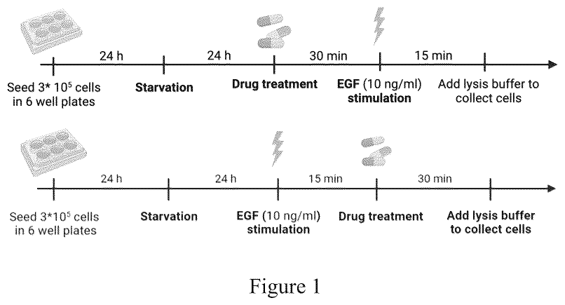

### DETAILED DISCRIPTION OF THE INVENTION

The following embodiments are made to clearly exhibit the above-mentioned and other technical contents, features and effects of the present invention. As the contents disclosed herein should be readily understood and can be implemented by a person skilled in the art, all equivalent changes or modifications which do not depart from the concept of the present invention should be encompassed by the appended claims.

Unless otherwise defined, all technical and scientific terms used herein have the same meaning as commonly understood by those skilled in the art to which the present invention pertains. It should be understood that the terminology used herein is for the purpose of describing specific embodiments only, and is not intended to be limiting.

As used herein, the singular forms “a”, “an” and “the” include plural references unless explicitly indicated otherwise. Thus, for example, reference to “a sample” includes a plurality of such samples and their equivalents known to those skilled in the art.

As used herein, the term “subject” refers to any warm-blooded species, including humans or non-human animals. Examples of the non-human animals include pet animals, domestic animals, and animals for competition, such as nonhuman primates, sheep, dogs, cats, horses, cows chickens, amphibians, reptiles, etc. Except when noted, the terms “patient” or “subject” are used interchangeably. A preferable subject is a human. The subject, such as a human, to be treated according to the present invention may in fact be any subject of the human population, male or female, which may be divided into children, adults, or elderly. Any one of these patient groups relates to an embodiment of the invention. The animal

According to the present invention, the combination markers expressed on PDAC-derived extracellular vesicles have the potential to serve as candidate markers for PDAC-associated EVs. Furthermore, PCP exhibits significant anticancer and anti-metastatic effects, achieved by influencing membrane receptors to decrease the secretion of cancer cell-derived CD9+ EVs and inducing alterations in the surface markers expressed on these EVs. Additionally, PCP plays a crucial role in inhibiting EGFR homodimerization and heterodimerization, making it a novel and promising therapeutic strategy for cancer patients.

### Prochlorperazine (PCP)

Prochlorperazine (PCP) is a phenothiazine derivative that has fewer extrapyramidal side effects on the central nervous system and has been used in regular clinical practice as an antiemetic rather than an antipsychotic. Previous studies have demonstrated the pivotal roles played by phenothiazine derivatives in the field of anticancer treatments and their ability to overcome drug resistance [3]. Recently, PCP also has been reported to act as an endocytosis inhibitor, which can modulate EGFR distribution and overcome anti-EGFR monoclonal antibody resistance [4], suggesting that PCP could affect the cell membrane activity to alter the location of membrane proteins.

### Pancreatic Ductal Adenocarcinoma (PDAC)

Pancreatic cancer, mainly known as pancreatic ductal adenocarcinoma (PDAC), is the fourth leading fatal neoplasm, with a trend of deterioration in the next decade. Given that the surgery is difficult to achieve, systemic chemotherapy is usually regarded as the first-line treatment and the combination of various chemical drugs. However, the above-mentioned treatment methods cannot solve the problem of poor prognosis in PDAC patients, and the five-year survival rate after treatment is only 10%. Thus, a new therapeutic strategy is urgently needed for PDAC, especially for drug development efforts directed toward patients with metastatic PDAC using biomarker-guided therapy.

### The Role of Integrin α6/β4 and CD9 in PDAC Metastasis

The extensive desmoplastic reaction is a prominent characteristic of PDAC, creating a physical barrier that hinders drug delivery. Activated pancreatic stellate cells (PSCs), influenced by growth factors released by PDAC cells, contribute to the dense deposition of extracellular matrix (ECM) molecules. This enhances drug resistance to standard therapies by fostering interactions with tumor cells. Integrin α6/β4, functioning as transmembrane protein receptors with integrin α6 and integrin β4 subunits, play a crucial role in cellular interactions. They selectively recognize the ECM component laminin and transduce crucial cell signaling upon binding with laminin. Previous studies have demonstrated that the cell surface glycoprotein and tetraspanin CD9 play an important role in mediating tumor-stroma cross-talk, thereby aggravating the fibrosis barrier in PDAC [2].

### Tumor-Associated Extracellular Vesicles (EVs) and Cancer

Extracellular vesicles (EVs) are small membrane-bound vesicles released by cells into the extracellular space, containing lipids, nucleic acids, and proteins. These components function as autocrine and/or paracrine factors, facilitating local and systemic cell-cell communication. Tumor-associated EVs can promote angiogenesis, facilitate cancer cell migration, induce epithelial-mesenchymal transition (EMT), and enhance drug resistance, thereby fostering aggressive cancer progression. Notably, they contribute to the establishment of pre-metastatic niches, influencing both tumor cells and the microenvironment at distant metastatic sites, thus creating a conducive milieu for the development of secondary malignant tumors. A prior study by Hoshino, A., et al. [5] highlighted the diversity of integrin heterodimeric combinations in tumor-associated EVs, elucidating their role in organ-specific metastasis. Therefore, tumor-associated EVs emerge as potential biomarkers and druggable targets in novel therapeutic strategies.

### The Promising Roles of Tumor-Associated EVs as Biomarker for PDAC

A cancer biomarker is a substance or process that indicates the presence of cancer in the body. Recently, GPC1 Exo-mRNA and tMV-mProtein, as a dual-biomarker, have underscored the potential of vesicular GPC1 expression for early PDAC screening and chemotherapy prognosis [6]. This highlights the utility of GPC1 antibodies for selecting PDAC patients and guiding treatment strategies.

### Head and Neck Squamous Cell Carcinoma (HNSCC)

Head and neck squamous cell carcinoma (HNSCC) is the sixth most common cancer worldwide with an increasing trend of its incidence. Alcohol consumption, smoking, and viral infections, such as the mucosal high-risk (HR) human papillomaviruses (HPVs) are major risk factors for HNSCC development. Surgery alone and radiotherapy alone can provide similar oncologic control and improved long-term survival rates in approximately 70 to 90% of patients with early-stage disease. In 2019, anti-programmed death-1 (PD-1) immune checkpoint inhibitors pembrolizumab on or after platinum-containing chemotherapy was approved for first-line treatment of patients with recurrent or metastatic HNSCC. The second-line treatment landscape has witnessed the evaluation of various options, including methotrexate, taxanes, cetuximab, and afatinib. Monotherapy studies focusing on these treatments have reported similar overall survival rates, ranging from 5 to 8 months. Unfortunately, due to the absence of efficacious therapies, the prognosis for patients with R/M HNSCC beyond the initial systemic treatment remains extremely poor.

### Non-Small Cell Lung Cancer (NSCLC)

Lung cancer, including in Taiwan, is the leading cause of cancer-related deaths globally. It can be broadly categorized as small cell lung cancer (SCLC) and non-small cell lung cancer (NSCLC), with NSCLC constituting 80% of lung cancer cases. NSCLC refers to any epithelial lung cancer type other than SCLC. The most common NSCLC subtypes are squamous cell carcinoma, large cell carcinoma, and adenocarcinoma, although there are other less frequent types, including rare histological variants. NSCLC typically exhibits lower sensitivity to chemotherapy and radiation therapy compared to SCLC. Surgery or surgery followed by chemotherapy may provide a potential cure for patients with resectable disease. Despite advances in diagnosis and treatment, the overall 5-year survival rate for lung cancer remains poor, with less than a 15% survival rate. Conventional therapies such as chemotherapy and radiotherapy often yield unsatisfactory outcomes in lung cancer patients, highlighting the significant clinical need for addressing drug resistance.

### Epidermal Growth Factor Receptor (EGFR)

EGFR is a prototypical receptor tyrosine kinase that is overexpressed in multiple cancers including PDAC, HNSCC, and NSCLC. EGFR belongs to the HER/ERBB family (consisting of HER/EGFR/ERBB 1 to 4) of receptor tyrosine kinases (RTKs), the activation of which leads to proliferation and metastasis of malignant cells and increased angiogenesis. Depending upon the type of ligand and the EGFR dimerization partner, several different signal transduction pathways can be engaged. The best studied pathways include the Ras/Raf/MEK/ERK and PI3K/PDKI/AKT pathways. Targeting EGFR has been a highly pursued strategy in cancer treatment over the past three decades. From extensive research and development, two fundamental approaches have proven to be effective. One approach involves the utilization of small molecule TKIs that specifically bind to the ATP-binding site in the tyrosine kinase domain of EGFR. Currently, three anti-EGFR TKIs, namely erlotinib, gefitinib, and lapatinib, have received FDA approval for use in oncology. The second approach employs monoclonal antibodies that target the extracellular domain of EGFR, effectively blocking the binding of natural ligands. These strategies have demonstrated significant potential in combating cancer.

### The Epidermal Growth Factor Family of Receptor Tyrosine Kinases (ERBBs)

Members of the ERBB tyrosine kinase family present some of the most altered proteins in cancer, and aberrant tyrosine kinase activation through gene alterations can drive tumorigenesis, tumor growth and progression. Oncogenic alterations of genes encoding members of the ERBB family, leading to aberrant ERBB signaling and driving tumor growth, have been reported in various types of cancer including breast, lung, head and neck, brain and gastrointestinal cancers. This family consists of four members that belong to the ERBB lineage of proteins (ERBB1-4). The ERBB proteins function as homo and heterodimers. EGFR and other cell-surface receptors (e.g., integrins) have pivotal roles during development and tumorigenesis. Behind the scenes of signaling cascades initiated by activated receptors, endocytosis determines the fate of internalized proteins through degradation in lysosomes or recycling them back to the cell surface or trans-Golgi network (TGN). The ERBB receptors are characterized by their defining feature: they are type 1 single membrane-spanning tyrosine kinase (RTK), which dimerize to initiate a host of signaling cascades which, ultimately, converge to promote cell growth, migration, and differentiation, and if persistently activated, can lead to cancer.

### Human Epidermal Growth Factor Receptor 3 (HER3)

As a unique member of the HER family, HER3 (ERBB3) lacks or has little intrinsic tyrosine kinase activity. It frequently co-expresses and forms heterodimers with other receptor tyrosine kinases (RTKs) in cancer cells to activate oncogenic signaling, especially the PI3K/Akt pathway and Src kinase. Overexpression of HER3 is known to correlate with poor prognosis for head and neck cancer [7]. Nowadays, there are no FDA-approved HER3 small molecule inhibitors due to its lack of tyrosine kinase domain activity. Therefore, inhibiting HER3 activation could be a distinct mechanism to suppress the formation of its heterodimers with other RTKs.

In the present invention, it has revealed that the combination markers expressed on PDAC-derived extracellular vesicles have the potential to serve as PDAC-associated extracellular vesicles. These combination markers may hold significant diagnostic and prognostic value in the context of PDAC, offering valuable insights into the development of targeted therapies and precision medicine approaches for PDAC patients.

In addition, it was found that PCP showed anticancer and anti-metastatic effect of PDAC, HNSCC, and NSCLC cells.

In addition, it was ascertained in the present invention that PCP affected the membrane receptors to alter the expressions of the surface marker expressed on PDAC cells and their derived extracellular vesicles.

In addition, it was also ascertained in the present invention that PCP suppressed the secretion of CD9+ EVs in PDAC cells.

In addition, it was also ascertained in the present invention that PCP affected the membrane receptors to halt the EGFR homodimerization and heterodimerization in PDAC, HNSCC, and NSCLC cells.

The present invention is further illustrated by the following examples, which are provided for the purpose of demonstration rather than limitation.

### EXAMPLES

### Materials and Methods

### Cell Lines and Cell Culture

The human pancreatic cancer cells (PANC-1 cells) were cultured in Dulbecco's modified Eagle's medium (DMEM) supplemented with 10% fetal bovine serum (FBS: Invitrogen), 10,000 units penicillin, 10 mg streptomycin and 25 μg Amphotericin B per mL (Gibco™ #15240062). Other human pancreatic cancer cells, AsPC-1 cells and BxPC-3 cells, were both cultured in RPMI 1640 Medium (Gibco™ #A 1049101) supplemented with 10% fetal bovine serum (FBS: Invitrogen), 1% MEM Non-Essential Amino Acids Solution (Gibco™ #11140050), 1% sodium pyruvate (Gibco™ #11360070), 10,000 units penicillin, 10 mg streptomycin and 25 μg Amphotericin B per mL (Gibco™ #15240062). The human lung fibroblasts (WI-38 cells) were cultured in Minimum Essential Medium (MEM, Gibco, #11900024) supplemented with 10% fetal bovine serum (FBS: Invitrogen), 1% sodium pyruvate (Gibco™ #11360070), 10,000 units penicillin, 10 mg streptomycin and 25 μg Amphotericin B per mL (Gibco™ #15240062). The human pancreatic duct epithelial cells (H6C7 cells) were cultured in RPMI 1640 Medium (Gibco™ #A 1049101) supplemented with 10% fetal bovine serum (FBS: Invitrogen), 1% MEM Non-Essential Amino Acids Solution (Gibco™ #11140050), 1% sodium pyruvate (Gibco™ #11360070), 10,000 units penicillin, 10 mg streptomycin and 25 μg Amphotericin B per mL (Gibco™ #15240062). The human HNSCC cells, SAS was cultured in Dulbecco's Modified Eagle Medium/Nutrient Mixture F-12 (DMEM/F12, Gibco, #11320033) supplemented with 10% fetal bovine serum (HyClone, #SH3039603), 100 units/ml penicillin and 1 mg/ml streptomycin (Sartorius, #030311B). The HNSCC cells FaDu and HSC3 were cultured in Minimum Essential Medium (MEM, Gibco, #11900024) supplemented with 10% fetal bovine serum (HyClone, #SH3039603), sodium pyruvate 0.11 mg/ml (Gibco, #11360070), 100 units/ml penicillin and 1 mg/ml streptomycin (Sartorius, #030311B). The NSCLC cell lines CL141, H441, and A549, along with the lung epithelial cell line BEAS-2B, were all cultured in RPMI 1640 medium (Thermofisher #11875093) supplemented with 10% FBS, 1% PSA, 1%, L-glutamine, and 1% non-essential amino acids. All the cells were maintained in a cell incubator containing 5% CO₂ and cultured at 37° C. For subculture, the cells were de-attached by trypsin EDTA (Sartorius, #030515B), collected in 15 ml centrifuge tube, centrifuged at 1000 rpm for 3 minutes, and remove the supernatant, then, suspend the cells with 5 ml of fresh culture medium and count the cells through trypan blue (0.4%) staining and hemocytometer.

### Chemical Compounds

Prochlorperazine, thioridazine, gemcitabine, and afatinib were obtained from Sigma. For the experiments, stock solutions were prepared as follows: 50 mM prochlorperazine, 50 mM thioridazine, 5 mM gemcitabine, and 10 mM afatinib, all dissolved in dimethyl sulfoxide (DMSO; Sigma).

### In Vitro PCP Treatment and EGF Stimulation

Two sequential treatments were utilized: pre-treatment with PCP before EGF stimulation to mimic the effect of antipsychotic treatment, and post-treatment with PCP after EGF stimulation to mimic tumor growth with drug treatment (Fingue 1).

For proximity ligation assay and western blotting of EGF stimulation and prochlorperazine treatment, cells were seeded 6×10⁴ on coverslips in 24-well plates for proximity ligation assay; cells were seeded 3×10⁵ on 6 well plates for western blotting, while incubated overnight to reach 80% confluent density before undergoing serum starvation for 24 h. Cells were then treated with prochlorperazine (5-15 μM) 30 min. DMSO was used as a vehicle control. For EGF stimulation, cells were incubated with 10 ng/mL with continuous drugs treatment for 15 min at 37° C.

### In-Situ Proximity Ligation Assay

Cells were seeded on coverslips in 24 wells and treated with PCP for 30 minutes before EGF (10 ng/ml) stimulation for 15 minutes. They were then fixed in 4% paraformaldehyde for 10 minutes at room temperature. The cells were permeabilized with 0.5% TritonX-100 in PBS for 10 minutes at room temperature. After permeabilization, the cells were incubated with a blocking solution (OLINK Bioscience) for 20 minutes at room temperature. Next, the cells were incubated with primary antibodies diluted in 1× antibody diluent (OLINK Bioscience) for 1 hour. Two primary antibodies, such as anti-EGFR mouse monoclonal antibodies and anti-p-EGFR rabbit polyclonal antibodies, were used at a 1:400 dilution. Afterward, the cells were incubated with the PLA anti-Mouse PLUS probe and anti-Rabbit Minus probe (OLINK Bioscience) as secondary antibodies, which contained specific oligonucleotides. This incubation step lasted for 1 hour at 37° C. Following that, the ligation reagents (OLINK Bioscience) were added and incubated for 30 minutes at 37° C. The ligation reagents consisted of 5× ligase buffer and 1× ligase, which were diluted with ddH2O. Finally, the amplify reagents (OLINK Bioscience) were added and incubated for 100 minutes at 37° C. The amplify reagents included 5× DUO Far red and 1× polymerase, which were diluted with ddH2O. After the incubation, the cells were mounted using an appropriate mounting medium. The images of the cells were acquired using a Zeiss 900 confocal microscope and quantified with Meta Morph. For each slide with an in-situ PLA sample, images were acquired at 3 different fields with 1 z-axis image. These images were then analyzed using ZEN image analysis software, which automatically counts the number of blobs per cell.

### Tumoroid Formation and Detection

Cancer cells were cultured into tumoroids for 7 days. Gemcitabine and PCP were added alone or in combination to the culture medium for 3 or 7 days, as indicated. The Cyto3D® Live-Dead Assay Kit and CellTiter-Glo® 3D Cell Viability Assay were utilized to evaluate the effectiveness of drug treatment. Immunofluorescence analysis was conducted to evaluate protein expression. The images were quantified by normalizing protein expression to nuclear staining with Hoechst.

### Collection of the Conditioned Medium

Seeded 5×10⁶ cells on 15 cm dish (Corning) and incubated for 2 days. Until the cells grew almost full of the dish, rinsing the dish twice with PBS to remove residual medium and replaced it with serum-free medium. 150 ml of serum-free medium was harvested twice after 24 hr and 48 hr and the total volume of conditioned medium is 300 ml. After collecting the conditioned medium, the cells and cell debris were removed by centrifugation at 500×g for 5 min and 2000×g for 20 min successively. The supernatant was stored at −20° C. for further isolation.

### Isolation and Purification of Extracellular Vesicles Secreted by Donor Cells

In the present invention, extracellular vesicle purification was performed using four different approaches: Tangential Flow Filtration (TFF), Total Exosome Isolation (TEI), and size-selective ultrafiltration.

### Tangential Flow Filtration (TFF)

The conditioned medium was filtered using a 0.22 μm filter prior to TFF purification. (The 0.8 μm filter would be replaced if we hoped the final products were microvesicle-enriched samples.) In the step of TFF purification, we used the sterilized hollow fiber modified Polyethersulfone (mPES) column (#D02-E500-05-N) (Spectrum Labs, Rancho Dominguez, CA, USA) and purified it using the mini-MAP.03 cross flow system (LefoScience, Taiwan). For the first use, the TFF column was washed with 0.22 μm-filtered Milli-Q water and rinsed with PBS to balance the system until pH 7.2-7.4; for the reuse column, the TFF column was washed with 0.1 N NaOH before washing 0.22 μm-filtered MilliQ water and rinsed with PBS to balance the system until pH 7.2-7.4. Then, the sample was put into the column and started condensing the sample. When the sample was condensed to 10-20 ml, the diafiltration step required the use of at least 5 volumes of PBS to exchange buffer and remove additional protein. Finally, we could collect the concentrated extracellular vesicles sample.

### Total Exosome Isolation (TEI)

After the purification of TFF, we used Total Exosome Isolation to obtain higher concentrated EV samples for further analysis. EVs are isolated by using Total Exosome Isolation Reagent (from cell culture media) (Invitrogen™ #4478359) following the manufacturer's instructions. Briefly, the EV samples (0.5 ml-1 ml) were mixed completely with TEI reagent and 10 incubated at 4° C. in a refrigerator overnight. Following incubation, EVs were complexed with TEI reagent and the supernatant was removed by centrifugation at 10,000×g for 60 minutes. Finally, the EV pellets were frozen at −80° C. in the refrigerator for longer storage.

### Size-Selective Ultrafiltration

The conditioned medium was filtered using a 0.22 μm filter prior to size-selective ultrafiltration (The 0.8 μm filter would be replaced if we hoped the final products were microvesicle-enriched samples.) In the step of size-selective ultrafiltration, we used the Amicon® Ultra-15 ml centrifugal filters (10 kDa/100 kDa MWCO, Millipore, Billerica, MA, USA) for the concentration. The concentrated EVs were washed at least 3 times with PBS to exchange the containing buffer. When the sample was condensed lower than 1 ml, we could collect the concentrated EV sample and aliquot it into several tubes. For the reuse centrifugal tubes, the tubes were washed with 0.1 N/0.5 N 0.22 μm-filtered NaOH buffer and rinsed with PBS to balance the system until pH 7.2-7.4 for the next isolation.

### Immunoblot Analysis of Cell Proteins and EV Proteins

Cells or EVs were lysed in RIPA lysis buffer (containing 50 mM Tris-HCl, 1% NP-40, 150 mM NaCl, 0.1% SDS, 1 mM PMSF, 1 mM Na₃VO₄) with complete protease and protease inhibitor cocktail (1:1000) (Sigma-Aldrich). The protein quantitation is analyzed by comparing to 2 mg/ml Bovine serum albumin (BSA). Prepare the cell sample with one-tenth of dilution and the EV sample without dilution. Next, prepare all sample to 30 ul then put 10 μl each well in 96-well plates. Protein concentrations were then determined using the BCA assay kit (Thermo, USA) according to the manufacturer's instructions. Then, the same amounts of protein lysates from cells and EVs were loaded onto 8-12% SDS polyacrylamide gels and subjected to electrophoresis at 80-100 V, followed by transferring the proteins to 0.22 or 0.45 μm poly-vinylidene fluoride membranes (Millipore). The membranes were blocked with 5% skimmed milk in PBST (Tris-buffered saline containing Tween-20) for 1 h at room temperature and then washed 3 times (10 minutes for each time) with TBST (1M Tris-HCL pH7.5, NaCl, Tween 20 in ddH2O). After the step of washing, probed with antibodies specific for EV-enriched markers at 40° C. overnight. Signals from HRP-coupled secondary antibodies were visualized using the enhanced chemiluminescence (ECL) detection system (Millipore #WBKLS0500). The membrane was exposed using enhanced chemiluminescence reagents and analyzed using the Fujifilm LAS4000 luminescent image analysis system. The following is the table of the antibody list used in the present invention.

### EV Size and Number Analysis by Nanoparticle Tracking Analysis (NTA)

The size distribution and concentration of isolated vesicles were assessed using the NanoSight NS300 instrument (Malvern Instruments Ltd, Malvern, UK), which was equipped with a 488 nm laser and a CCD camera (model NS-300). The obtained data were analyzed using the Nanoparticle Tracking Analysis (NTA) software, specifically versions 3.1 builds 3.1.46. To ensure accurate measurements, the samples were diluted 10-1000 times with sterile and filtered phosphate-buffered saline (PBS) in order to reduce the number of particles in the field of view below 150 per frame. Readings were captured for a duration of 60 seconds at a rate of 25 frames per second (fps), with the camera level set to 15 or 16. Additionally, temperature monitoring was performed manually.

### Transwell Migration Assay

In the transwell migration assay, cells were seeded in upper chambers of Transwell plates (Transwell® Permeable Support, 6.5 mm insert, 24 well plate, 8.0 μm pore polyester membrane with sterile, Corning, #3464) in serum-free (SF) medium and seeded 5×10⁴-1×10⁵ cells per well, while lower chambers were added medium with 10% FBS as an attractant. Drugs were treated in both upper and lower chambers, and incubated on the 24-well plate at 37° C. for 16-24 hours. The cells were fix with 4% paraformaldehyde (PFA, SIGMA, #F8775) for 10 minutes and staining with 1% crystal violet (MERCK, 1 g Crystal violet powder dissolved in 75 ml ddH₂O and 25 ml menthol then filters by 0.45 μm filter) for 0.5 hours. After drying the insert completely, take at least 3 cell pictures randomly in every sample by microscope and count the number of cells by Image J.

### Wound Healing Migration Assay

The culture inserts (ibidi, #80209) was put on 6 well dishes (2 inserts for each well), irradiated UV for 20 minutes and cells were seeded into the well of the insert and incubating 24 hours. The insert was removed gently and washed twice with PBS to remove the old medium and cell and be observed the gap which was formed by the insert through the microscope and captured the picture of the gap at 0 hours. After I captured of the gap, different treatments were added and incubated for 16 hours. The result was observed by the microscope and quantified by Image J.

### Sulforhodamine B Colorimetric (SRB) Assay

The cell viability detection and cell proliferation rate were determined by sulforhodamine B(SRB) assay based on the measurement of cellular protein amount. The following experimental methods have been optimized for screening compounds of adherent cells in 96-well plate format. Cancer cells were seeded 3,000 cells per well for 24-72 hours, then treated with different concentrations of drug for 24, 48 and 72 hours, respectively. On time points 24, 48 and 72 hours, discard the medium, and cells were fixed by cold 10% trichloroacetic acid (SIGMA) at 4° C. for 1 hour or overnight. After fixation, plates were washed twice with clean water and air-drying. After drying, cells were stained with 0.1% SRB solution 100 μl/well (in 1% acetic acid) at room temperature for 1 hour and then washed with cold 1% acetic acid until the excess dye is melted out. Waiting for full dryness, adding 100 μl of 20 mM Tris-base to each well, and the plate was shaken for 10 seconds and detected at OD 510 nm by the ELISA reader (TECAN Sunrise™). Cell viability was normalized to control and draw the image by Prism 8.

### EV Detection by Total Internal Reflection Fluorescence (TIRF) Microscope

The antibody combination of EGFR/GPC1/EpCAM was used for targeting extracellular vesicles derived from pancreatic cancer and was incorporated into a biochip, which was then incubated at 4° C. overnight. Subsequently, the EV samples were diluted to a concentration of 1E+10 particles/ml and added to the biochip containing the antibodies. After a one-hour incubation at room temperature, fluorescent antibodies, specifically FITC-anti-CD9 and APC-anti-integrin α6, were introduced and incubated for an additional hour at room temperature. Finally, the images were analyzed using a TIRF microscope.

### EV Detection by Nanoflow Cytometry (NanoFCM)

EV samples containing 3×10⁹ particles were incubated with diluted antibodies (1:1000), including FITC-anti-CD9 antibodies, PE-anti-CD63 antibodies, and APC-anti-integrin β4 antibodies, for 30 minutes in a 37° C. water bath. Following the incubation period, an Amicon® Ultra Centrifugal Filter with a 100 kDa molecular weight cutoff (MWCO) was used and rinsed with 1× PBS buffer. The samples were centrifuged at 4000×g for 5 minutes, with the centrifugation process repeated three times. After centrifugation, the samples were retrieved and diluted 10-100 times for detecting the expression levels of EV surface proteins by NanoFCM. The results were analyzed using Kaluza Analysis Software.

### CD9/CD63 Protein-Blocking Assay

The utilization of anti-CD9/CD63 antibody-conjugated magnetic beads (Miltenyi Biotec #130-110-913, #130-110-918) was further validated to assess the function of different EV subpopulations. Anti-IgG magnetic beads (Miltenyi Biotec #130-047-501) were used for the control group. BxPC-3 cell-derived secretomes were incubated with the anti-IgG/CD9/CD63 antibody-conjugated magnetic beads and then co-cultured with PANC-1 cells for 20 hours. A transwell cell migration assay was subsequently conducted to determine the function of different EV subpopulations.

### Statistical Analysis

All data are presented as mean±SEM and were analyzed using GraphPad Prism software (Version 6.0). p values were calculated using one-way analysis of variance with a post hoc Bonferroni test with >3 replicates for multiple comparisons and using unpaired Student's t test for two groups.

### Results

1. PCP inhibited the protein expression of EGFR downstream signaling pathways in HNSCC, NSCLC, and PDAC cells.

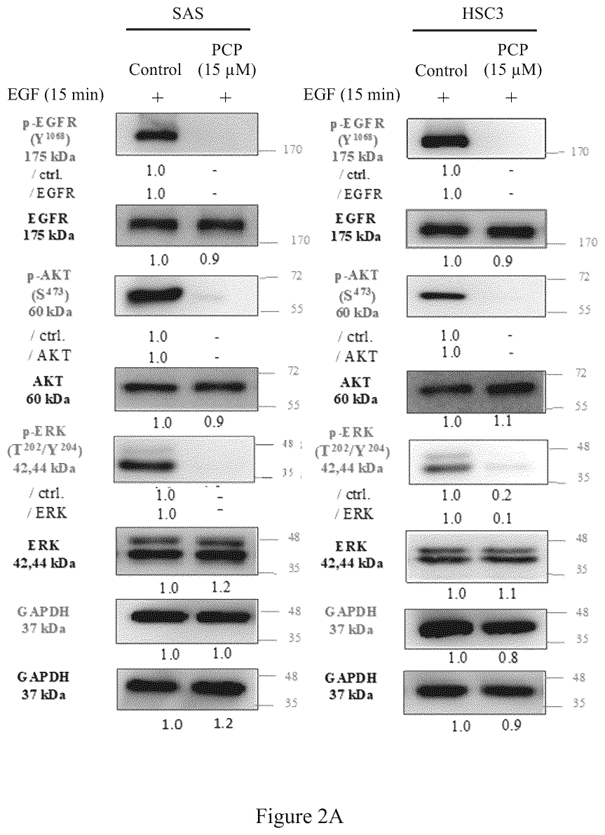

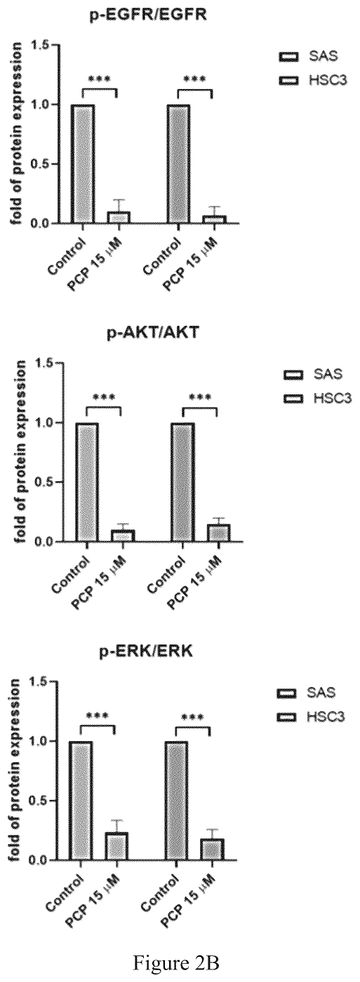

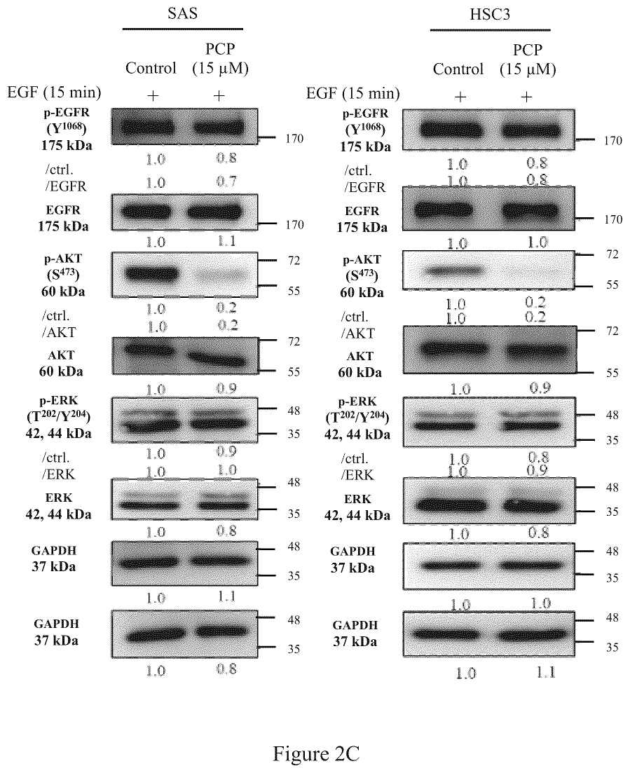

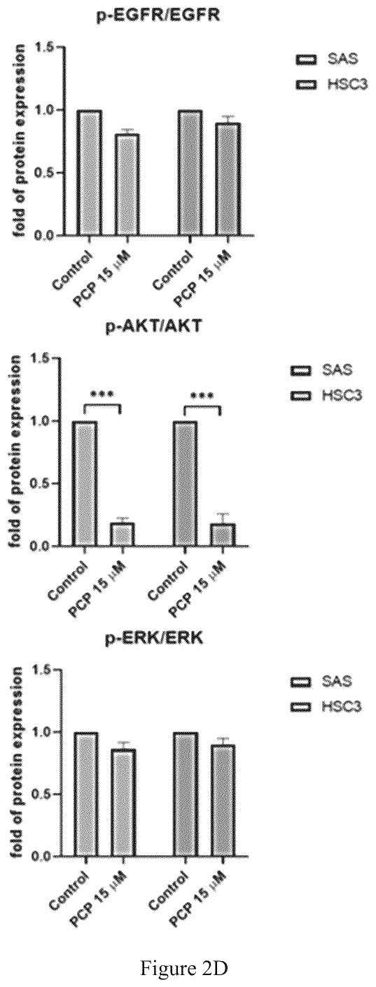

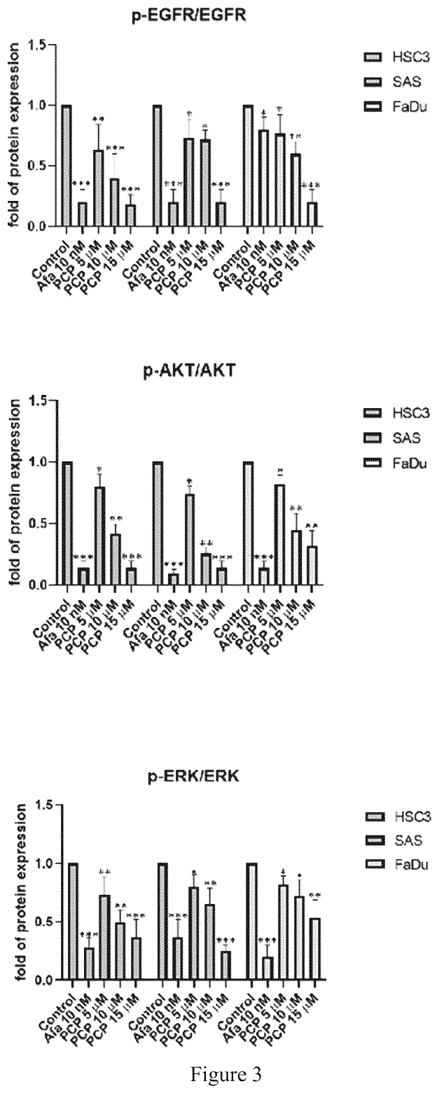

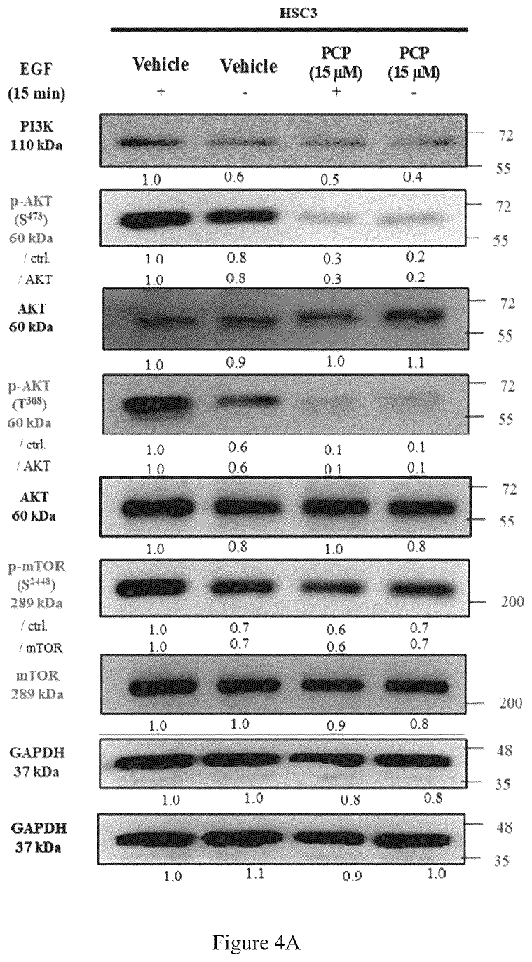

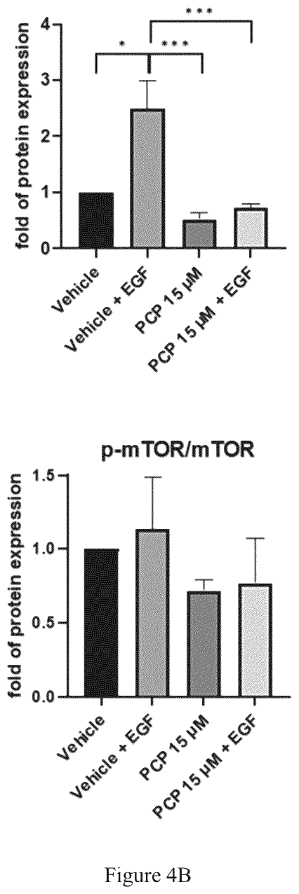

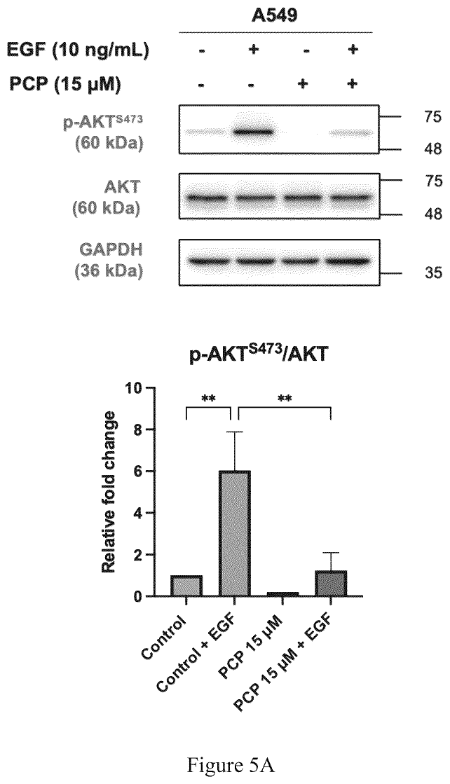

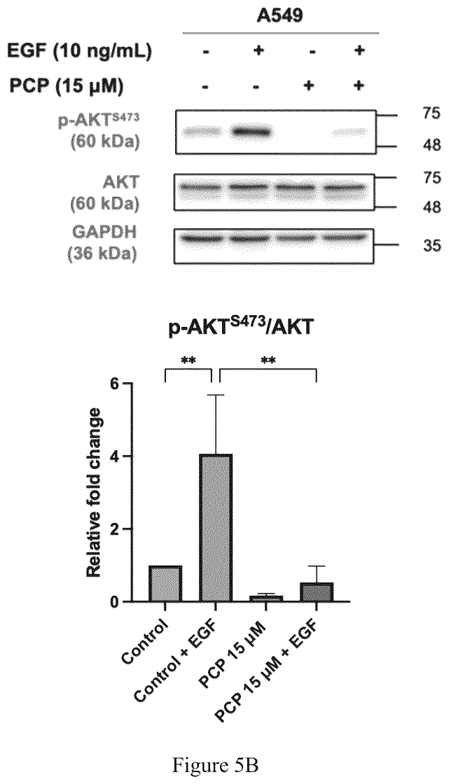

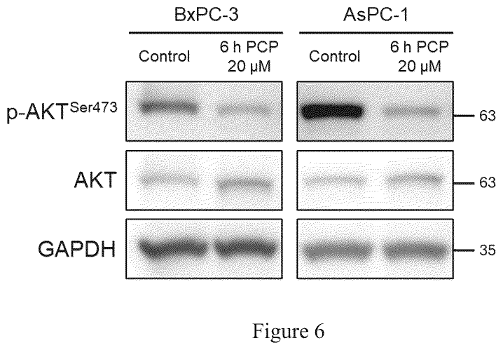

In previous studies, it was established that PCP could inhibit EGFR endocytosis and inhibit EGFR activation to improve drug efficacy of EGFR monoclonal antibody cetuximab [4]. However, our findings elucidate the inhibitory effect of PCP on EGFR signaling pathways under various sequential treatment conditions. Two sequential treatments were utilized: pre-treat with PCP before EGF stimulation to mimic the effect of antipsychotic treatment, and post-treat with PCP after EGF stimulation to mimic tumor growth with drug treatment (FIG. 2). The results unequivocally demonstrated the effectiveness of pre-treatment with PCP in inhibiting EGF-induced EGFR signaling pathway activation, showcasing its ability to specifically target the pathway rather than directly suppressing activated EGFR. Western blotting analysis further revealed a dose-dependent downregulation of EGFR/MAPK/ERK and PI3K/AKT/mTOR protein expression (FIG. 3 and FIG. 4). Importantly, our data indicated that post-treatment alone was unable to inhibit EGFR activation and the MAPK pathway. However, AKT activation could be suppressed by both sequential treatments. Additionally, the inhibitory effect of PCP on AKT activation was also observed in NSCLC and PDAC cells (FIG. 5 and FIG. 6).

2. Thioridazine treatment suppressed EGFR downstream activation in HNSCC cells.

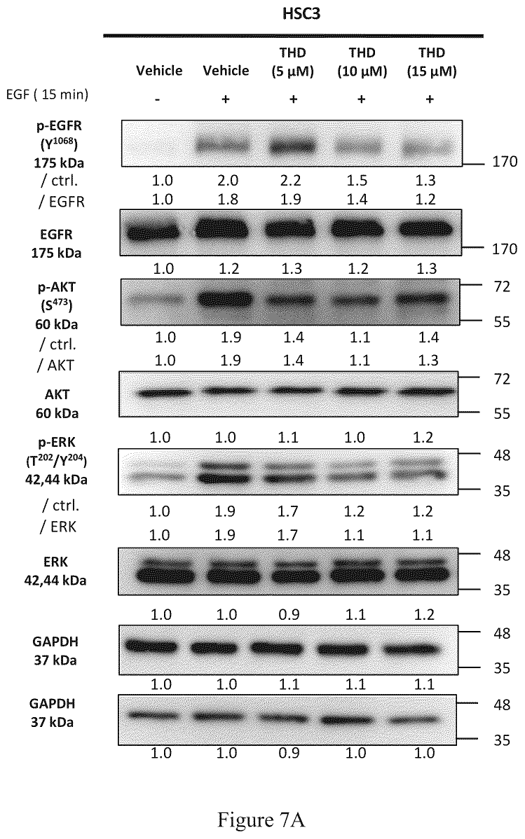

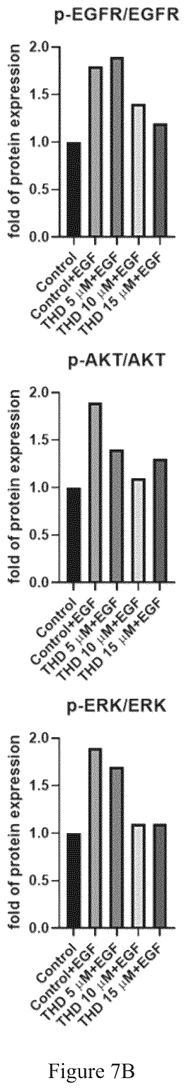

Thioridazine (THD), an analog of PCP, belongs to the phenothiazine derivatives drug class. Interestingly, our observations indicate that thioridazine exhibits inhibitory effects on EGFR downstream pathways, including ERK and AKT activation in HSC3. However, it is worth noting that the extent of inhibition on AKT activation by thioridazine is not as potent as that observed with PCP. These findings shed light on the potential of THD as a therapeutic agent in modulating EGFR downstream pathways (FIG. 7).

3. PCP inhibited the dimerization of ERBB family members, including EGFR homodimerization and heterodimerization, in HNSCC, NSCLC, and PDAC cells.

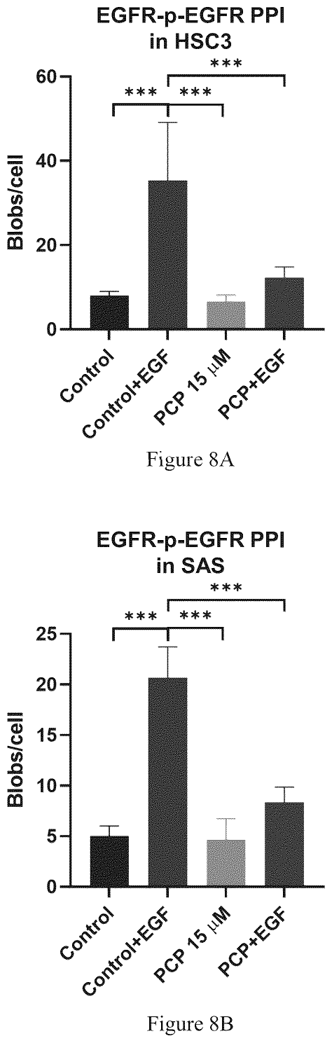

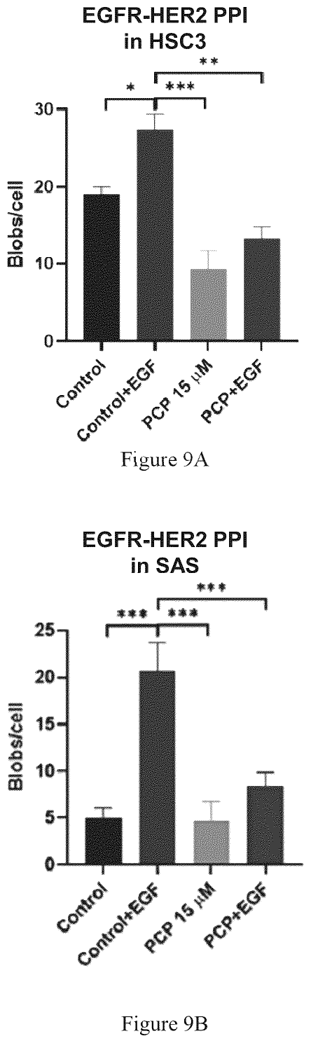

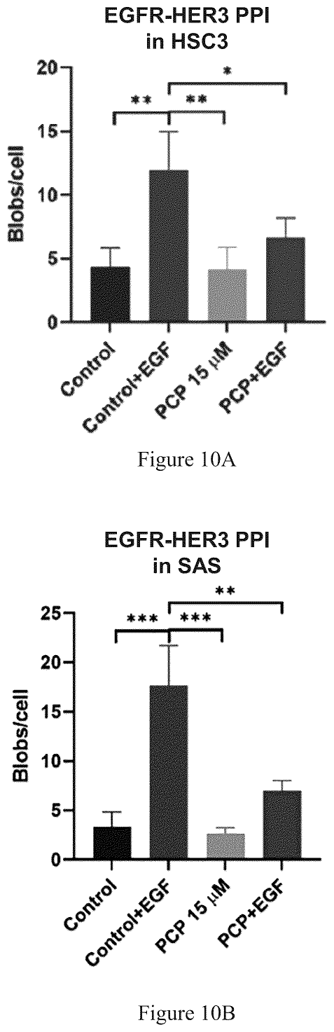

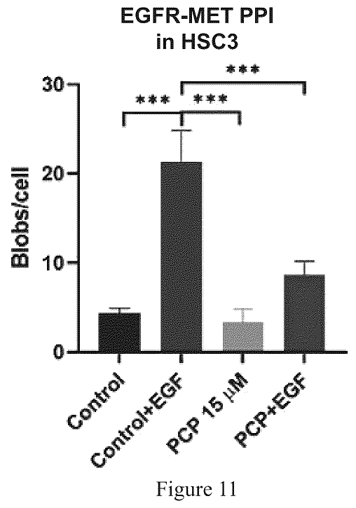

Based on the above results, we recognized that PCP may employ a different mechanism compared to EGFR TKIs, as it did not directly inhibit the tyrosine kinase domain. Therefore, we hypothesized that PCP might exert its effects by influencing the dimerization of EGFR, leading to the suppression of its activation. We examined the effect of PCP on EGFR homodimerization and heterodimerization with HER2, HER3, and MET in HNSCC cells by proximity ligation assay. Notably, our results demonstrated that PCP treatment effectively suppressed EGFR-p-EGFR protein-protein interactions, indicating its ability to disrupt EGFR homodimerization in HSC3 and SAS cells (FIG. 8), aligning with our earlier findings. Furthermore, we observed that PCP also exhibited the capability to inhibit EGFR heterodimerization involving HER2, HER3, and MET in both HSC3 and SAS cells (FIGS. 9-11).

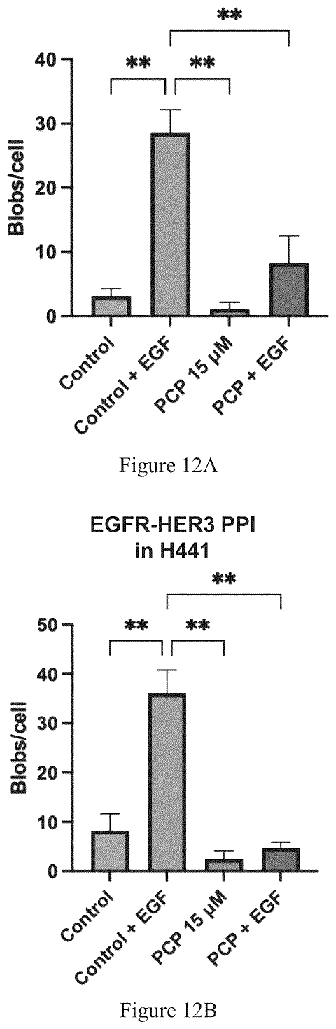

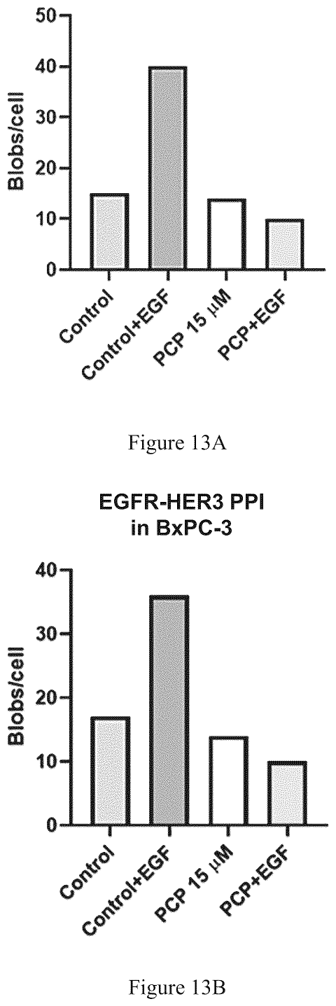

Chemotherapy resistance poses a significant hurdle in the treatment of metastatic cancers, such as lung cancer and pancreatic cancer, remaining a major clinical challenge. Non-small cell lung cancer (NSCLC) and pancreatic ductal adenocarcinoma (PDAC) commonly exhibit high expression levels of EGFR. Therefore, the combined effect of EGFR homodimerization and heterodimerization can intensify tumor aggressiveness and contribute to resistance against therapies. Research findings have demonstrated an upregulation of EGFR, HER2, and HER3 expression in gemcitabine-resistant models of NSCLC and PDAC. Furthermore, the administration of an antibody mixture targeting all three receptors inhibited tumor growth in mice and led to the downregulation of the ERBB receptors [8]. Therefore, our objective was to expand the cancer type to examine the impact of PCP treatment on the ERBB receptors in both NSCLC and PDAC cells. The results showed that PCP pretreatment inhibited EGFR homodimerization and heterodimerization in both NSCLC and PDAC cells (FIG. 12 and FIG. 13), consistent with results observed in HNSCC cells. These findings demonstrate that PCP had the potential to affect the activity of the cell membrane in cancer cells, resulting in changes in the dimerization of ERBB family members.

4. PCP treatment alone and in combination with afatinib inhibited EGFR-HER3 protein-protein interactions, potentially overcoming afatinib resistance in HNSCC cells.

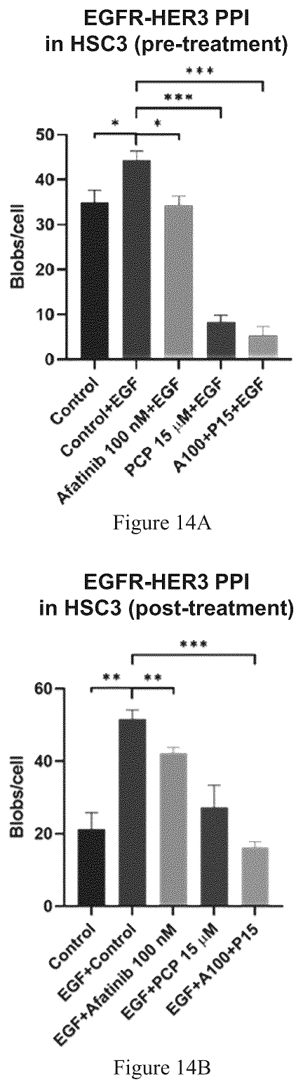

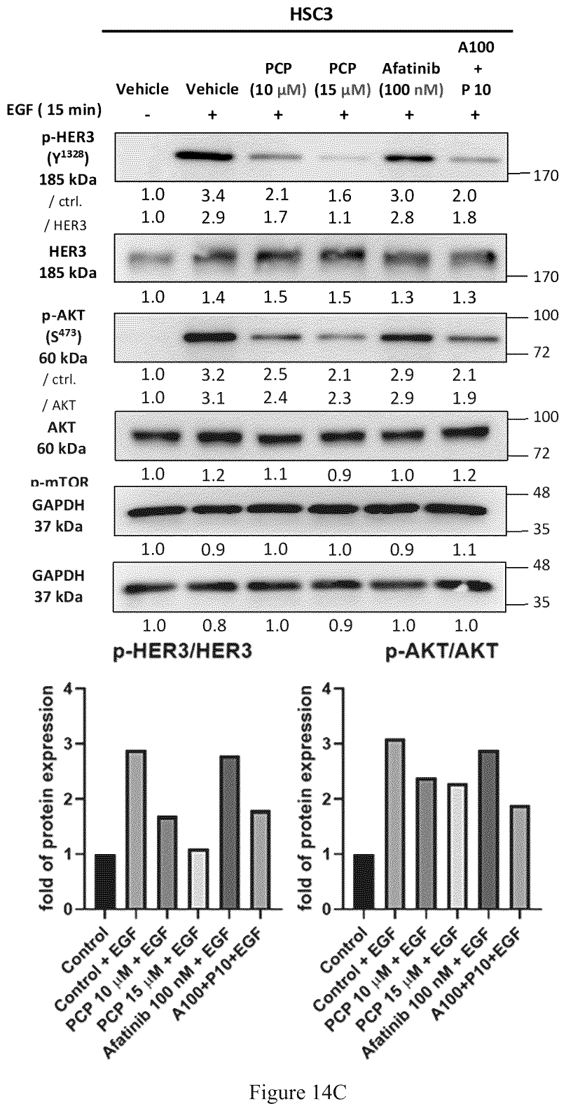

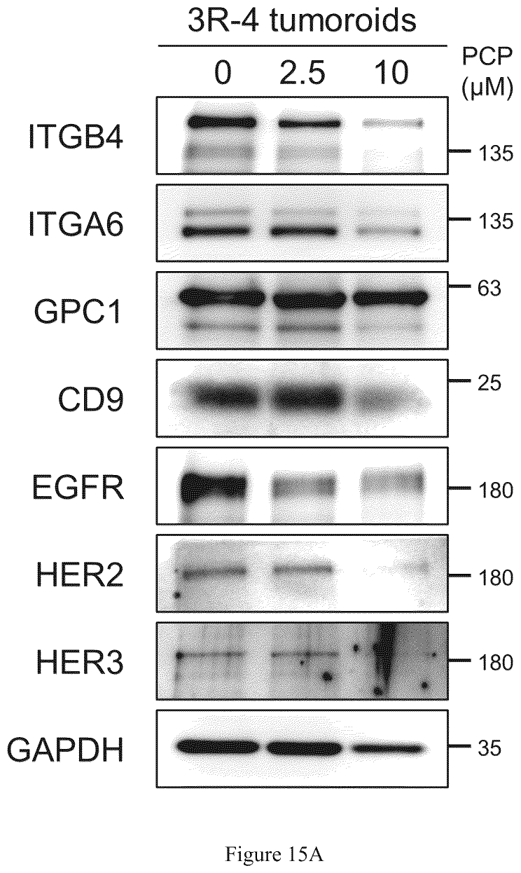

Building upon our previous findings, we identified that the inhibition of the PI3K/AKT/mTOR pathway might involve other upstream pathways, including various receptor tyrosine kinases. Motivated by these observations, we then designed experiments using two distinct treatment regimens, varying the order of EGF stimulation and PCP treatment, to investigate the impact on EGFR-HER3 protein-protein interactions. Our experimental results demonstrated that both pre-treatment and post-treatment with PCP effectively inhibited EGFR-HER3 protein-protein interactions (FIG. 14). These findings were particularly significant, underscoring the potential of PCP treatment in overcoming afatinib resistance in HSC3 cells. Notably, HER3, which has an impaired kinase domain, cannot be directly targeted by tyrosine kinase inhibitors. By inhibiting EGFR-HER3 protein-protein interactions and HER3/AKT signaling pathways, PCP offered a promising therapeutic strategy to overcome afatinib resistance in HNSCC. These results highlighted the potential of PCP as an alternative or complementary treatment option.

5. PCP treatment alone and in combination with gemcitabine inhibited metastasis, stemness, and epithelial-mesenchymal transition (EMT), potentially overcoming gemcitabine resistance in pancreatic cancer patient-derived cells and their cultured tumoroids.

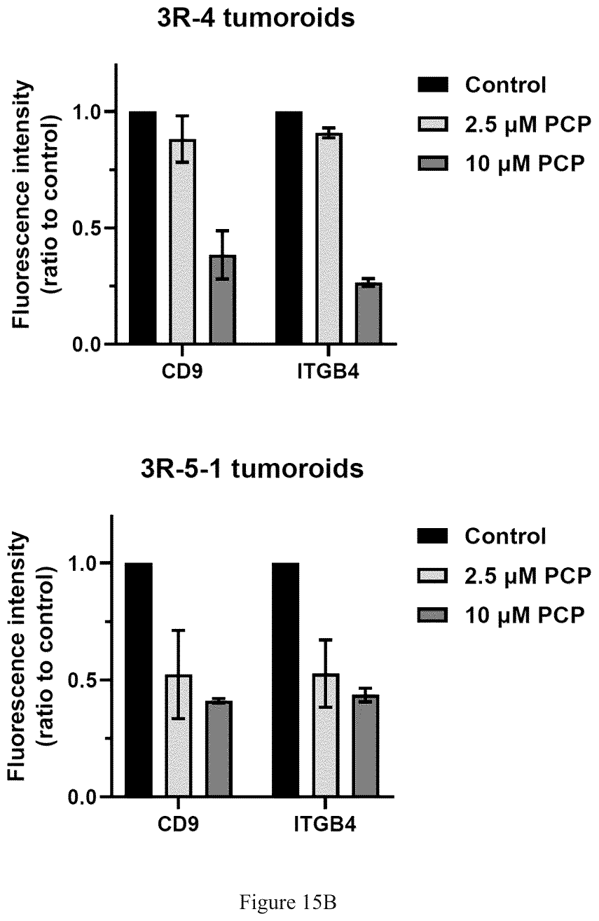

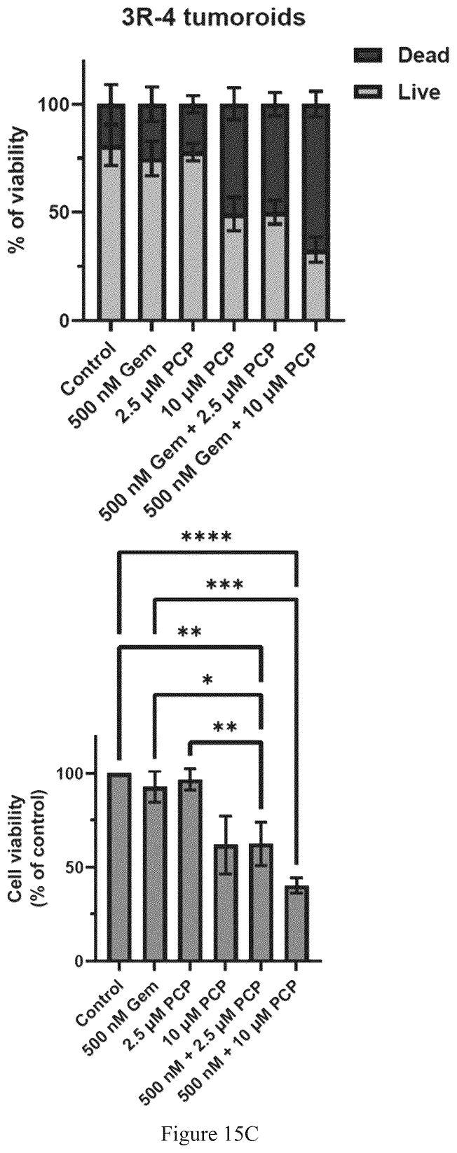

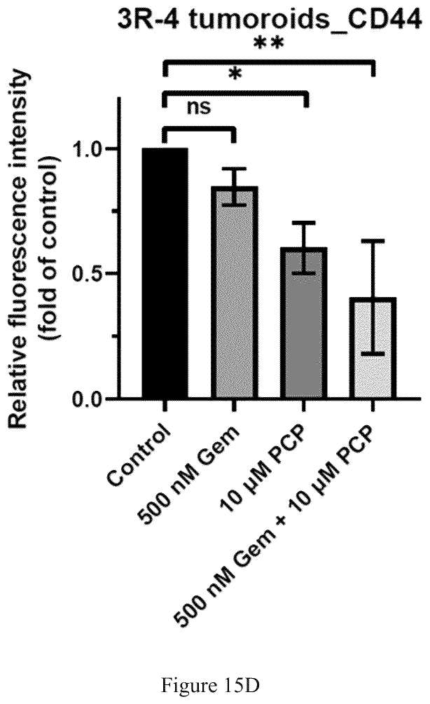

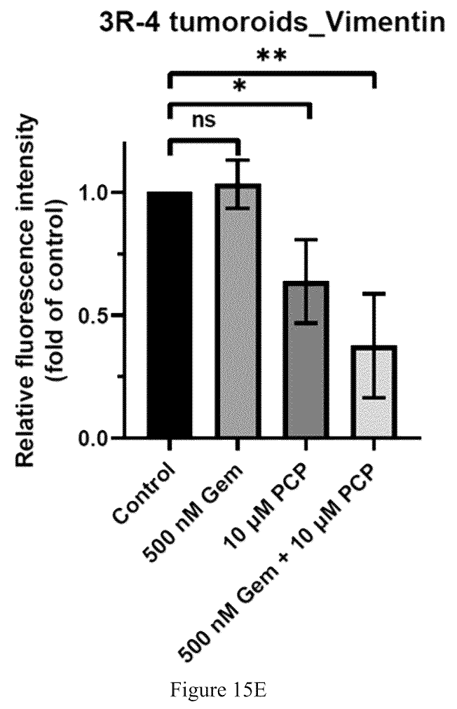

Building upon the aforementioned findings, we expanded our research to PDAC, which commonly exhibits high expression levels of EGFR. We identified pancreatic cancer patient-derived floating tumor cells from ascites and employed 3D tumoroid formation techniques for further clinical validation. Surprisingly, PCP not only suppressed EGFR dimerization but also inhibited EGFR expression levels (FIG. 15A). Additionally, PCP downregulated integrin α6/β4 and CD9 (FIGS. 15A and 15B), highlighting its effect in suppressing tumorigenesis and metastasis. However, the unchanged expression levels of GPC1 following PCP treatment highlighted the utility of the GPC1 antibody for the selection of PDAC patients.

The combination of gemcitabine and PCP treatment suppressed the expression of CD44 and vimentin, demonstrating significant anti-PDAC effects in pancreatic cancer patient-derived cells and their cultured tumoroids (FIGS. 15C-15E). These findings indicate that PCP could be repurposed as an innovative therapeutic strategy in combination with gemcitabine, offering a potential solution for overcoming challenges associated with chemotherapy resistance in pancreatic cancer.

6. PCP suppressed the pro-metastatic properties of PDAC-derived extracellular vesicles, thereby achieving anti-metastatic activity in PDAC cells.

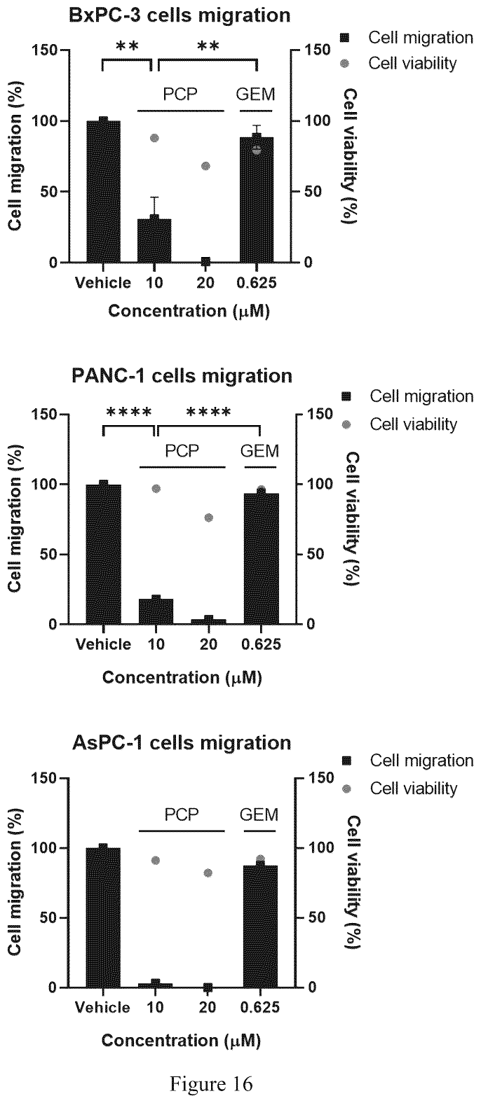

In the initial stages, gemcitabine was utilized to investigate its anti-metastatic activity compared to PCP treatment in PDAC cells. To initiate this exploration, a transwell migration assay in vitro was performed to assess the migration ability of PDAC cells. The results indicated a significant suppression of PDAC cell migration by PCP compared to gemcitabine treatment (FIG. 16). Next, additional investigations were conducted to assess whether PCP could inhibit the migratory capability of PDAC-derived secretomes. To achieve this, a co-culture cell migration system was established to examine the behavioral changes in normal cells treated with PDAC-derived secretomes.

For the cell line selection, the H6C7 cell line, derived from normal human pancreatic duct epithelial (HPDE) cells and immortalized, was employed to investigate the interactions between PDAC cells and human pancreatic duct cells. This cell line serves as a valuable tool for studying the molecular and cellular interactions that occur between PDAC cells and their surrounding pancreatic ductal epithelial cells. Additionally, to simulate the lung tissue environment and validate PDAC cells with high metastatic potential to the lung, human lung fibroblasts known as WI-38 cells were chosen as a relative normal cell model. By utilizing this model, the interactions between PDAC cells and the lung microenvironment can be examined and assessed.

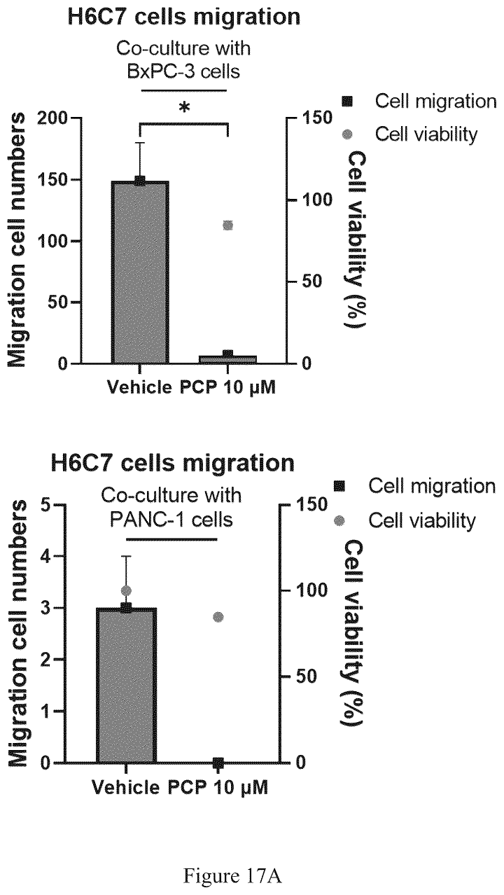

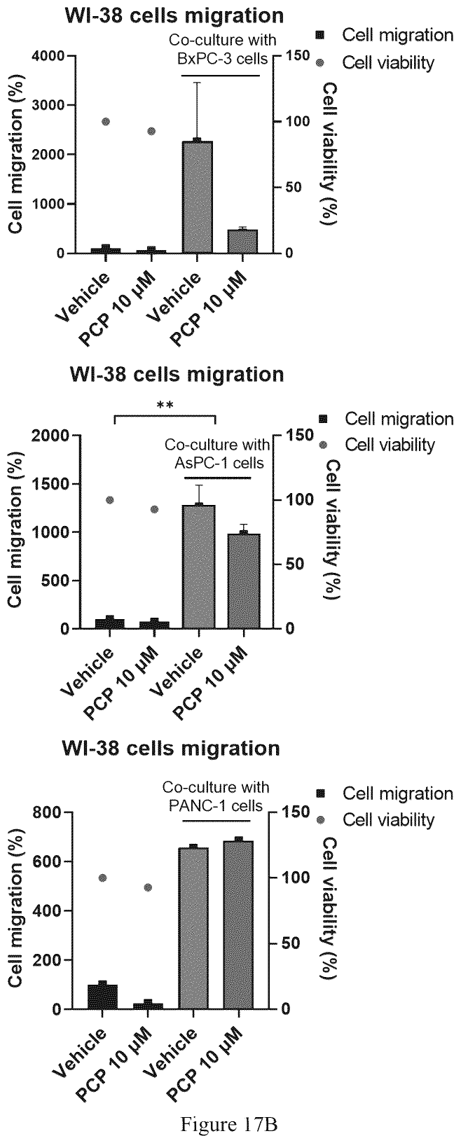

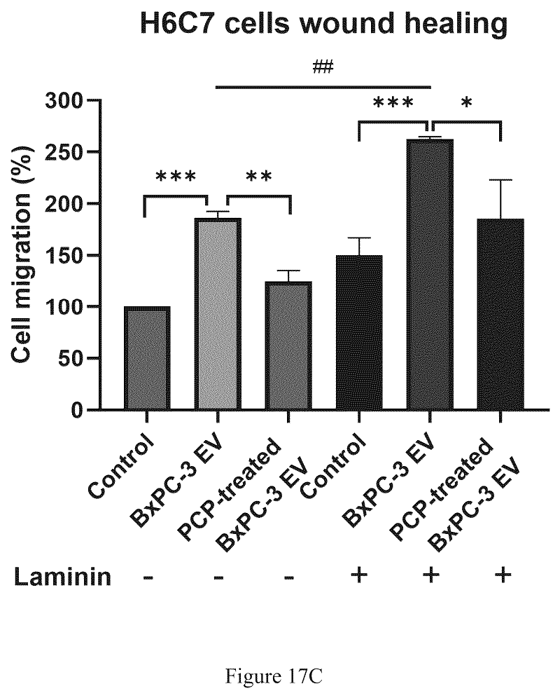

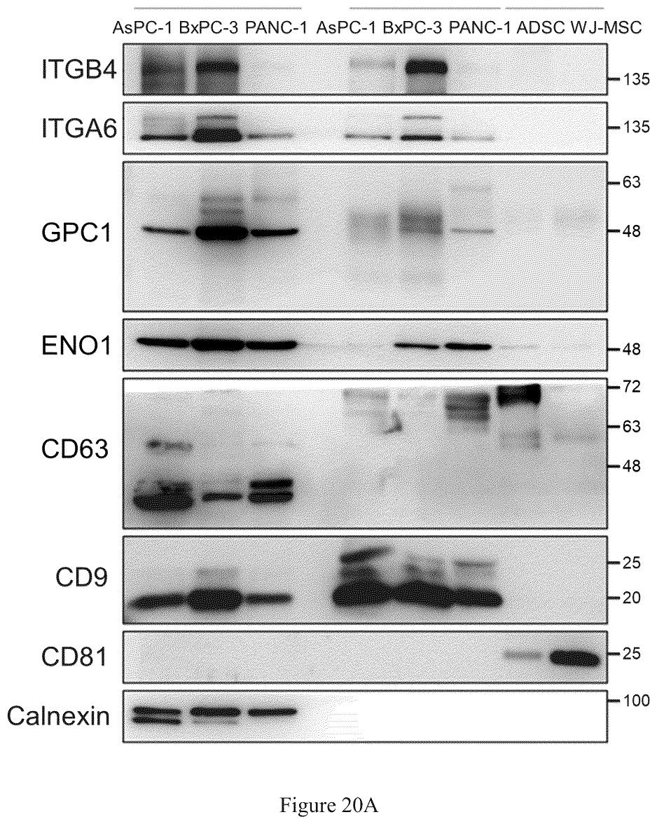

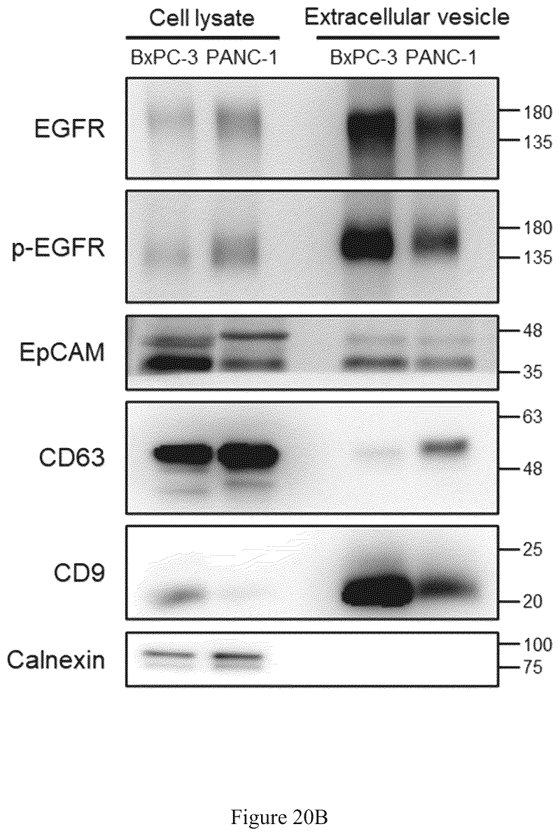

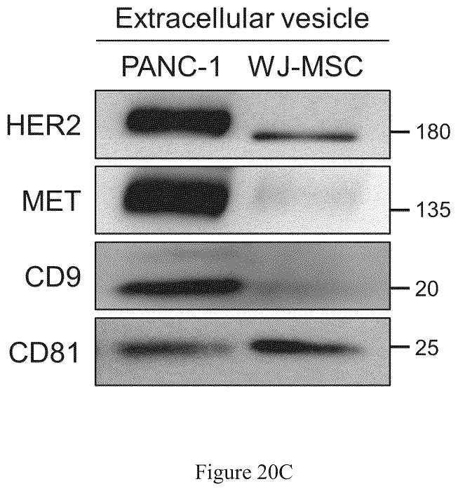

As the results shown in FIG. 17A, H6C7 cells significantly altered migratory behavior after being co-cultured with BxPC-3-derived secretomes for 4 days. However, PCP could significantly suppress the effect. However, in the co-culture system involving PANC-1-derived secretomes, this phenomenon was not found to be significant. For the lung microenvironment validation, WI-38 cells significantly altered migratory behavior after being co-cultured with PDAC-derived secretomes. However, the effect of PCP treatment varied among the three groups of PDAC-derived secretomes. PCP treatment showed the strongest inhibition of migratory capability in BxPC-3-derived secretomes, followed by a comparatively weaker effect in AsPC-1-derived secretomes, and the least effect in PANC-1-derived secretomes (FIG. 17B). This observation was consistent with the expression levels of integrin α6/β4 among these PDAC cell lines (FIG. 20A).

To investigate the potential of PCP treatment to inhibit the functionality of integrin α6/β4 on PDAC-derived EVs, a laminin coating assay was performed to assess the migration ability of H6C7 cells. In this assay, H6C7 cells were seeded in the wound healing inserts with or without 1 μg/cm² laminin coating in the six-well plate. Once the cells were attached, the wound healing inserts were removed, and H6C7 cells were exposed to BxPC-3-derived EVs and PCP-treated BxPC-3 cell-derived EVs. The results of the laminin coating assay revealed a significant increase in the migratory capacity of H6C7 cells when treated with BxPC-3-derived EVs. The result suggests that the integrin α6/β4 present on these EVs promoted the migration of H6C7 cells when exposed to a laminin-coated surface. However, when H6C7 cells were treated with EVs derived from PCP-treated BxPC-3 cells, the migratory capacity of the cells was significantly repressed (FIG. 17C). This indicates that PCP treatment may have an inhibitory effect on the functionality of integrin α6/β4 on PDAC-derived EVs, leading to a reduction in their ability to promote cell migration. These findings suggested that PCP treatment had the potential to disrupt the interaction between integrin α6/β4 on PDAC-derived EVs and laminin, thereby inhibiting the migratory capability of recipient cells. This further substantiates the hypothesis that PCP could function as a therapeutic agent targeting PDAC-derived extracellular vesicles and mitigating the metastatic potential of PDAC cells, particularly within the extracellular matrix-enriched tumor microenvironment.

7. PCP suppressed the pro-metastatic properties of NSCLC-derived extracellular vesicles, thereby achieving anti-metastatic activity in NSCLC cells.

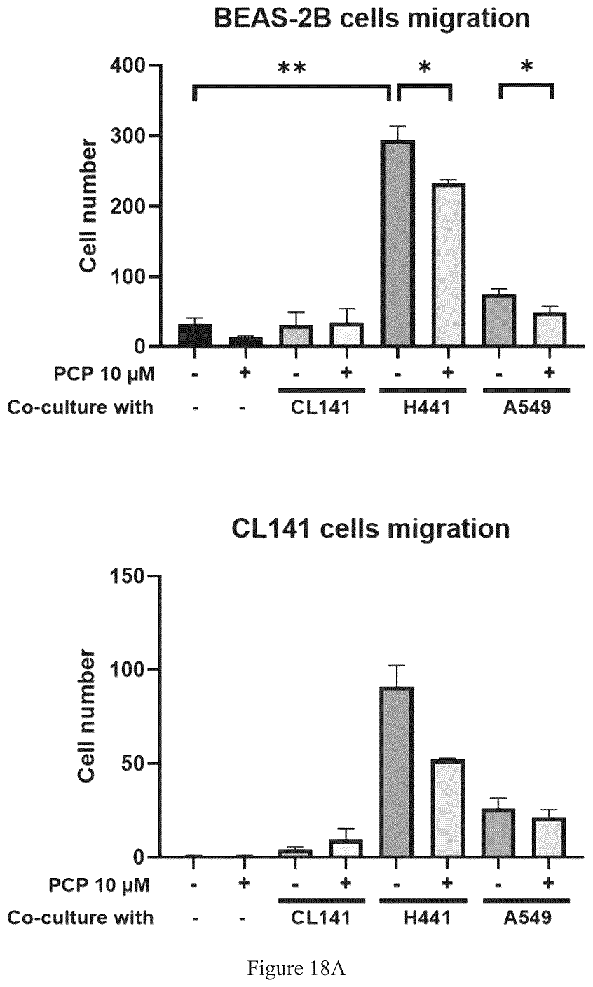

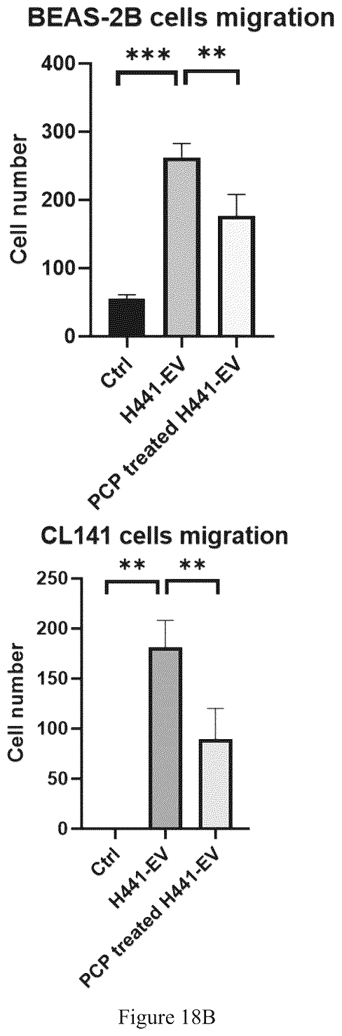

The effect of PCP on inhibiting the migratory properties of NSCLC-derived extracellular vesicles was assessed using a transwell cell migration assay in NSCLC cells (FIG. 18), with comparable results observed in PDAC cells.

8. Marker combinations such as EGFR, GPC1, EpCAM, CD9, and ITGA6 on pancreatic cancer-associated extracellular vesicles play a role in prognosis and guide PCP treatment strategies.

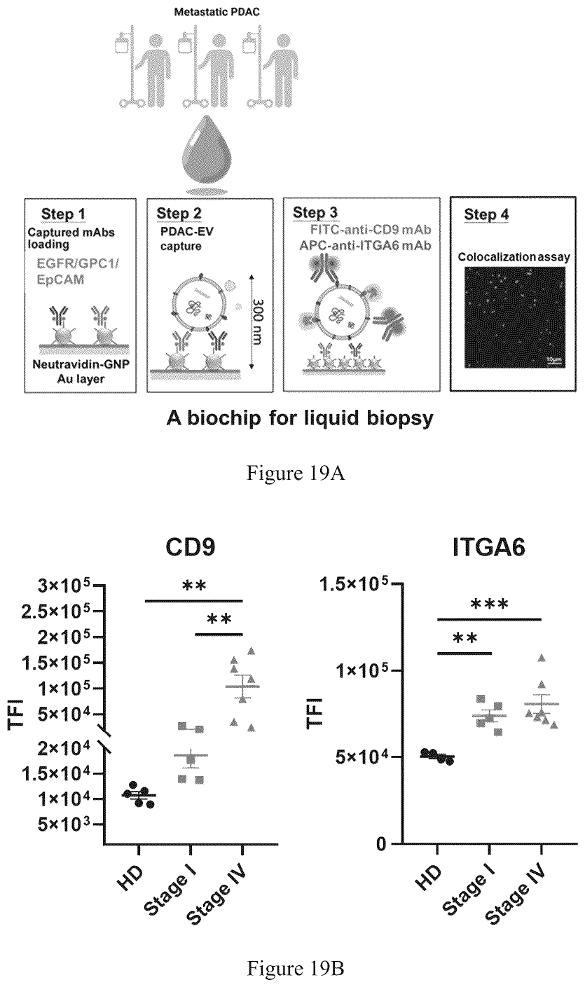

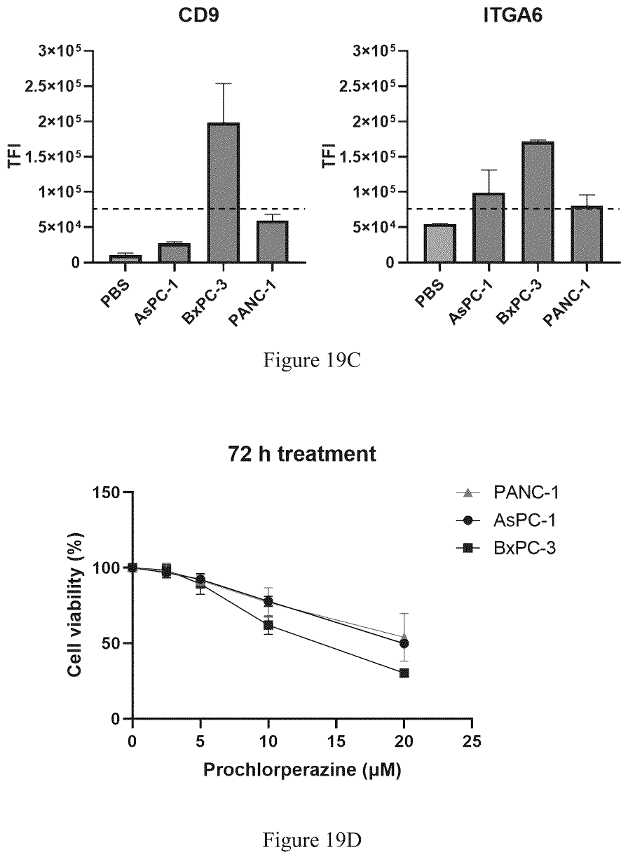

Utilizing EGFR/EpCAM/GPC1 antibody combinations to isolate PDAC-associated EVs could differentiate different stages of PDAC patients. We confirmed co-expression of CD9 and ITGA6 on these EVs in both PDAC cell lines and patient plasma samples (FIG. 19, IRB No. 2022-07-032BC) using TIRF technology. These data suggest the importance of CD9 and integrin α6/β4 (ITGA6/ITGB4) on the surface of tumor-associated EVs in PDAC metastasis. Results presented in FIGS. 19C and 19D indicate that BxPC-3 cell-derived extracellular vesicles exhibiting high expression levels of EGFR, GPC1, EpCAM, CD9, and ITGA6 may guide PCP treatment strategies and correlate with increased sensitivity to PCP in BxPC-3 cells.

9. Validation of surface markers, including integrin β4, integrin α6, GPC1, ENO1, EGFR, p-EGFR, EpCAM, HER2, and MET, on PDAC-derived extracellular vesicles.

In the present invention, EVs from three PDAC cell lines (AsPC-1, BxPC-3, and PANC-1) were isolated using TFF. To identify surface markers that can differentiate PDAC-associated extracellular vesicles from those derived from normal cells, mesenchymal stem cells (MSCs) were selected as the normal control. The validation process involved assessing surface marker expression from equivalent protein amounts on PDAC cells and their corresponding EVs via Western blotting analysis. The results revealed the expression of integrin β4, integrin α6, GPC1, ENO1, EGFR, p-EGFR, EpCAM, HER2, and MET, on PDAC-derived EVs (FIG. 19).

Furthermore, we compared the expression of surface markers between PDAC-derived EVs and MSC-derived EVs. Analysis revealed that PDAC-derived EVs exhibited higher expression levels of surface markers, including integrin β4, integrin α6, GPC1, ENO1, HER2, and MET, compared to MSC-derived EVs, demonstrating the potential of these markers for identifying PDAC-associated EVs (FIGS. 20A and 20C).

10. PCP inhibited laminin-induced cell proliferation in PDAC cells, highlighting the role of integrin α6/β4 in guiding PCP treatment for pancreatic cancer.

Firstly, we selected three PDAC cell lines for experimental validations: BxPC-3 cells with high expressions of integrin α6/β4, AsPC-1 cells with intermediate expressions of integrin α6/β4 and PANC-1 cells with low expressions of integrin α6/β4. The purpose of this selection was to investigate their drug sensitivity to PCP treatment. The result showed that BxPC-3 cells were significantly more sensitive to PCP treatment than AsPC-1 cells and PANC-1 cells (Table 1).

TABLE 1
The IC50 of PCP treatment in different pancreatic cancer cell lines.

| IC50 of PCP (μM) | 24 h | 48 h | | | | | 72 h | | |
|---|---|---|---|---|---|---|---|---|---|
| BxPC-3 | >20 | 17.9 ± 2.1 | | ** | | | 12.1 ± 1.7 | | ** |
| | | | {close oversize bracket} | | | | | {close oversize bracket} | |
| AsPC-1 | >20 | >20 | | | {close oversize bracket} | * | 19.9 ± 1.1 | | |
| PANC-1 | >20 | >20 | | | | | 15.4 ± 0.8 | | |

BxPC-3 cells were significantly more sensitive to PCP treatment than AsPC-1 cells and PANC-1 cells. Data values were measured at different time points as dosages. *, p<0.05 compared to control group. **, p<0.01 compared to control group.

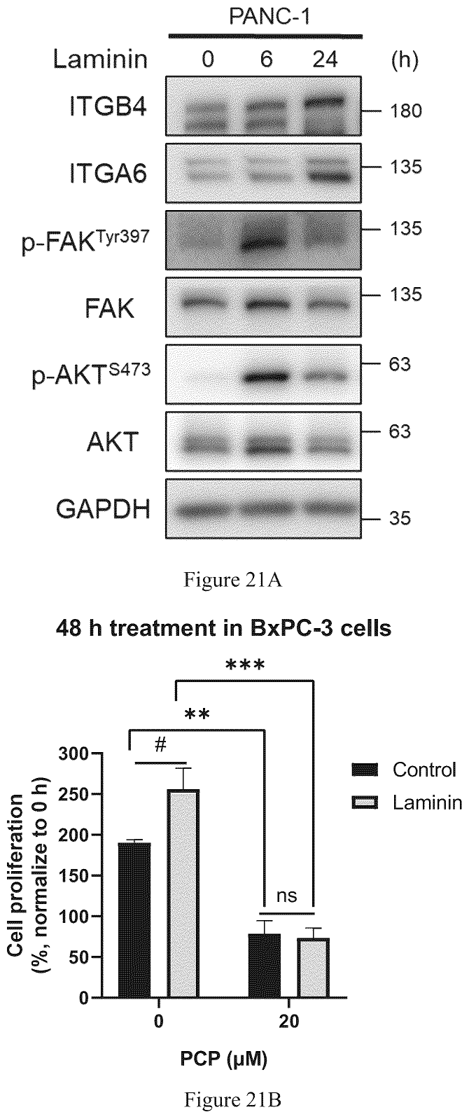

Secondly, to further validate that PCP may target integrin α6/β4 to achieve its anti-cancer activity, ECM ligand laminin was utilized to enhance the binding affinity of integrin α6/β4 in its extended-open (active, high affinity) conformation. When integrin α6/β4 binds to laminins, it undergoes activation and clustering, subsequently initiating the FAK/AKT downstream signaling pathway (FIG. 21A). Ultimately, this signaling cascade modulates cellular behaviors such as proliferation and migration. Following binding with 1 μg/cm² laminin for 48 hours, BxPC-3 cells exhibited a significantly increased cell proliferation rate (FIG. 21B). Additionally, PCP treatment reversed the increased cell proliferation rate observed in BxPC-3 cells following laminin binding (FIG. 21B). Therefore, PCP inhibited laminin-induced integrin α6/β4 activation, thereby suppressing PDAC cell proliferation and demonstrating the role of integrin α6/β4 in guiding PCP treatment for metastatic PDAC.

11. PCP inhibited integrin α6/β4 on PDAC cell-derived EVs within short-term of treatment.

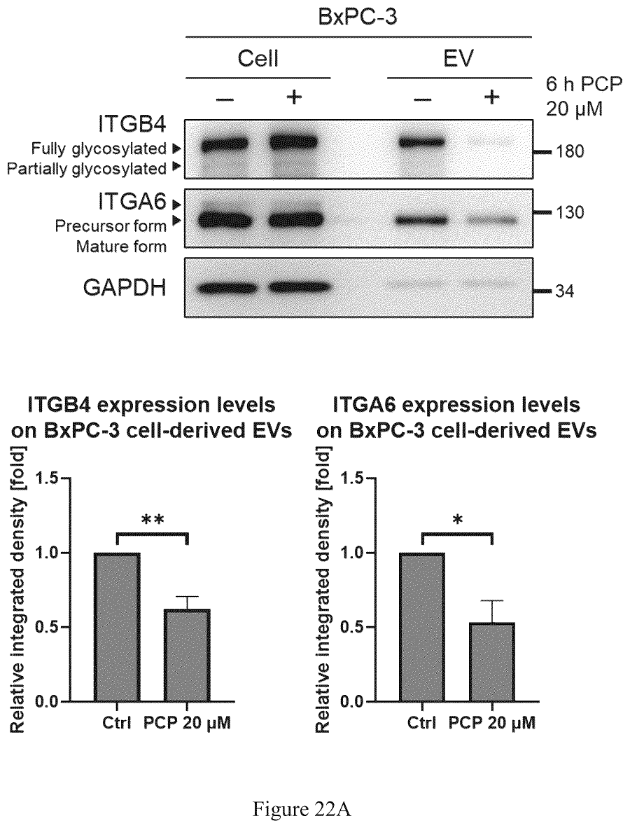

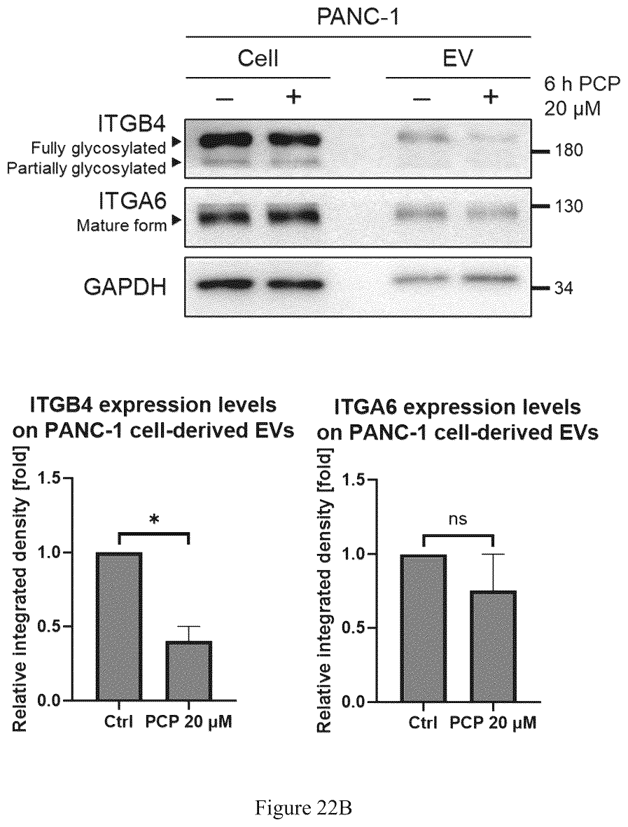

To investigate the sensitivity of PCP treatment in PDAC-derived EVs and validate integrin α6/β4 as a potential biomarker for guiding PCP treatment in pancreatic cancer, the expressions of integrin α6/β4 on PDAC-derived EVs were determined. Specifically, BxPC-3-derived EVs, which exhibit high integrin α6/β4 expressions, were chosen for comparison with PANC-1-derived EVs, known for their low integrin α6/β4 expressions. By examining the expression levels of integrin α6/β4 on PDAC-derived EVs, the potential effectiveness of PCP treatment could be evaluated. As the results shown in FIG. 22, short-term PCP treatment reduced integrin α6/β4 protein expressions on EVs from BxPC-3 and PANC-1 cells, with BxPC-3 showing higher PCP sensitivity than PANC-1. This finding highlights the potential of PCP treatment to disrupt the interaction between PDAC-derived EVs and the extracellular matrix, consequently impairing the migratory and invasive capabilities in PDAC cells.

12. PCP exhibited strong anti-metastatic effects by modulating the secretion of ITGB4+CD9+ EVs and downregulating the co-expression levels of CD9 and integrin β4 on EVs from BxPC-3 cells.

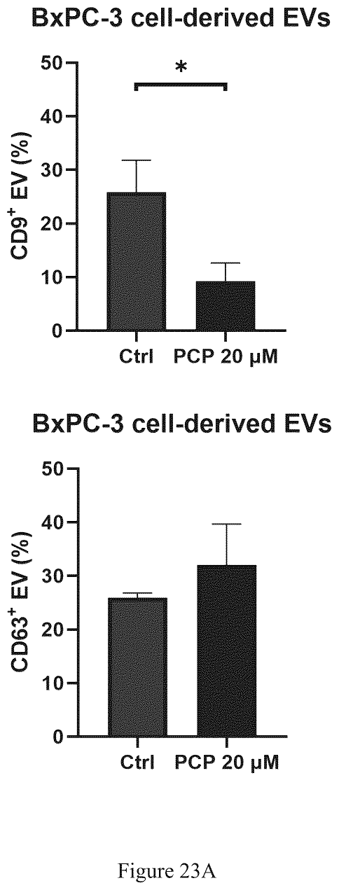

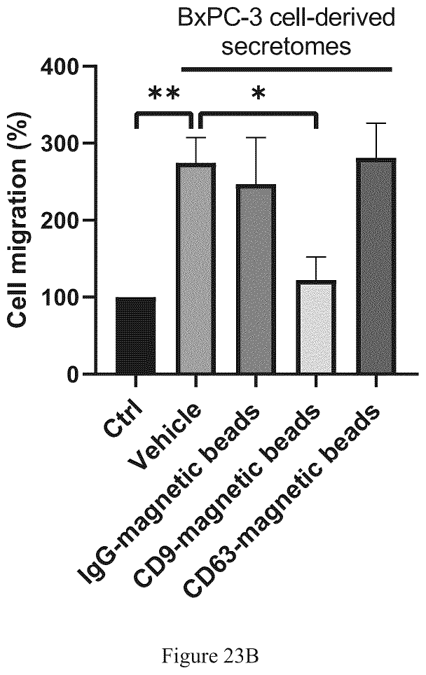

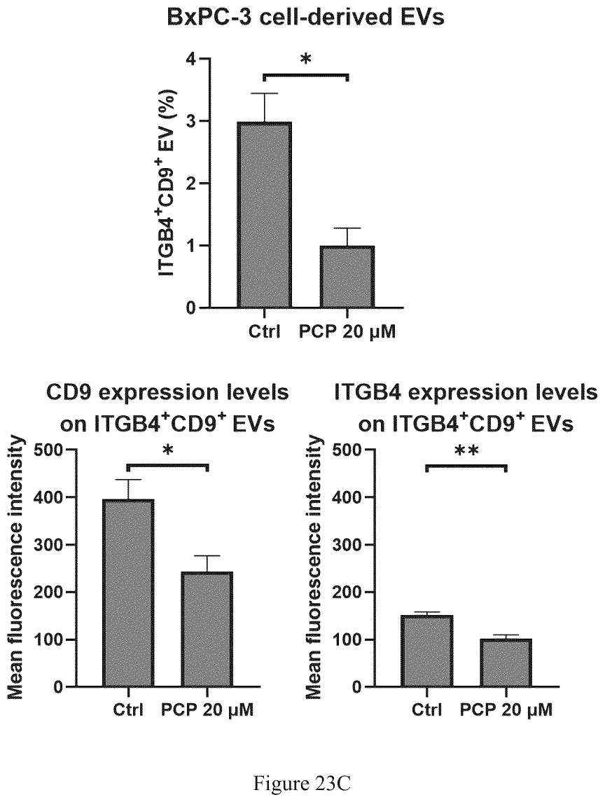

As depicted in FIG. 23A, PCP treatment significantly modulated the secretion of different EV subpopulations from BxPC-3 cells. The utilization of CD9/CD63-blocking antibody-conjugated magnetic beads further validated the function of these EV subpopulations, indicating that CD9 proteins expressed on BxPC-3 cell-derived EVs play a specific pro-migratory role in enhancing the migratory ability of PANC-1 cells (FIG. 23B). Furthermore, PCP treatment significantly suppressed the secretion of ITGB4+CD9+ EVs and inhibited the co-expression levels of CD9 and integrin β4 proteins on BxPC-3 cell-derived EVs (FIG. 23C). This highlights the benefits of utilizing PCP treatment to inhibit the pro-migratory capacity of PDAC-derived EVs and impede pre-metastatic niche formation, aiming to suppress PDAC progression.

13. Combination treatment of gemcitabine and PCP exerted a synergistic cytotoxicity effect on pancreatic cancer patient-derived cells and their cultured tumoroids in ex vivo drug testing.

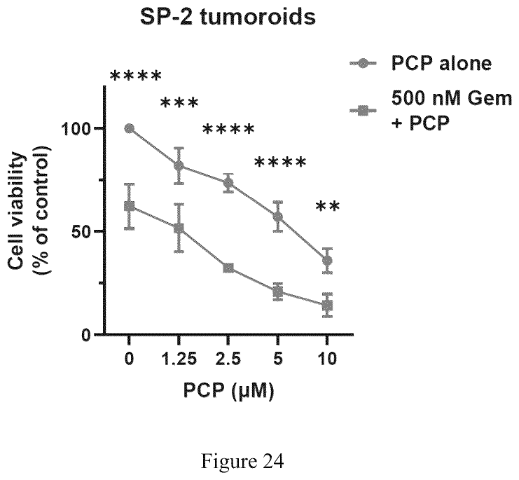

Utilizing 3D tumoroid formation techniques for further clinical validation, the combination treatment of gemcitabine and PCP demonstrated a synergistic cytotoxicity effect on pancreatic cancer patient-derived cells and their cultured tumoroids (FIG. 24), indicating that PCP could be repurposed as a novel therapeutic strategy in combination with gemcitabine for the treatment of pancreatic cancer.

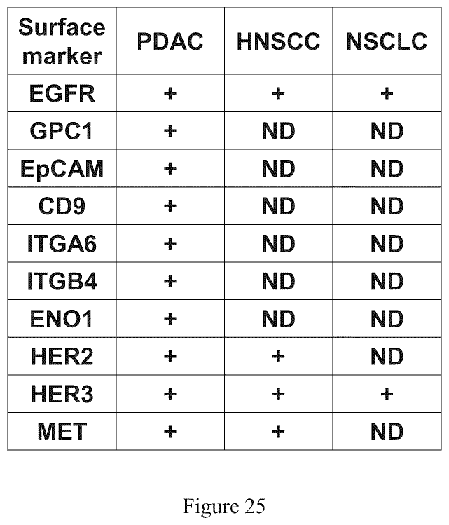

In view of the results, it could be concluded that PCP exerts its anticancer effects by impacting the membrane receptors of cancer cells, particularly by inhibiting the EGFR downstream signaling pathway. This is achieved through the suppression of EGF-induced EGFR homodimerization and heterodimerization in vitro. Furthermore, PCP demonstrates anti-metastatic effects by suppressing the pro-metastatic ability of PDAC and NSCLC-derived extracellular vesicles and down-regulating the expressions of surface markers. Our results present a novel and promising therapeutic strategy for patients with PDAC, HNSCC, and NSCLC. Notably, the treatment with PCP is more sensitive to cancer cells and their derived extracellular vesicles expressing high levels of CD9 and integrin α6/β4, leading to enhanced anti-metastatic effects and greater down-regulation of the corresponding integrin α6 and integrin β4 expressions. Absolutely, the expression levels of integrin α6 and integrin β4 on cancer cell-derived extracellular vesicles can serve as valuable biomarkers to guide PCP treatment in clinical applications. This precision medicine-based approach using PCP for various cancers allows for a targeted patient selection strategy, ensuring that individuals with specific biomarker profiles receive the most suitable and effective treatment (FIG. 25). The repurposing of PCP as an anti-metastasis drug holds great promise in the field of clinical oncology, offering new and potentially life-saving therapeutic options for patients with metastatic cancers. By tailoring treatment based on individual biomarker expression, the effectiveness of PCP in inhibiting cancer cell migration and metastasis can be maximized while minimizing potential side effects for patients who are most likely to benefit from the therapy. This personalized medicine approach represents a significant advancement in cancer treatment and provides hope for improved outcomes and better quality of life for cancer patients.

While this invention has been described in connection with what is presently considered to be practical exemplary embodiments, it is to be understood that the invention is not limited to the disclosed embodiments. On the contrary, it is intended to cover various modifications and equivalent arrangements included within the spirit and scope of the appended claims.

### REFERENCES

1. Wang, V. M., et al., CD9 identifies pancreatic cancer stem cells and modulates glutamine metabolism to fuel tumour growth. Nat Cell Biol, 2019. 21 (11): p. 1425-1435.
2. Nigri, J., et al., CD9 mediates the uptake of extracellular vesicles from cancer-associated fibroblasts that promote pancreatic cancer cell aggressiveness. Sci Signal, 2022. 15 (745): p. eabg8191.
3. Yeh, C. T., et al., Trifluoperazine, an antipsychotic agent, inhibits cancer stem cell growth and overcomes drug resistance of lung cancer. Am J Respir Crit Care Med, 2012. 186 (11): p. 1180-8.
4. Chew, H. Y., et al., Endocytosis Inhibition in Humans to Improve Responses to ADCC-Mediating Antibodies. Cell, 2020. 180 (5): p. 895-914 e27.
5. Hoshino, A., et al., Tumour exosome integrins determine organotropic metastasis. Nature, 2015. 527 (7578): p. 329-35.
6. Li, H., et al., Extracellular Vesicular Analysis of Glypican 1 mRNA and Protein for Pancreatic Cancer Diagnosis and Prognosis. Adv Sci (Weinh), 2024. 11 (11): p. e2306373.
7. Saddawi-Konefka, R., et al., HER2 and HER3 as Therapeutic Targets in Head and Neck Cancer. Cancer J, 2022. 28 (5): p. 339-345.
8. Rabia, E., et al., Anti-tumoral activity of the Pan-HER (Sym013) antibody mixture in gemcitabine-resistant pancreatic cancer models. MAbs, 2021. 13 (1): p. 1914883.

What is claimed is:

1. A method for treating a cancer with high expression of a specific marker in a subject, comprising the steps of
(a) collecting the cancer cell samples of the subject;
(b) determining if the specific marker is overexpressed on cancer cells or their extracellular vesicles (EVs); and
(c) administering to the subject a therapeutically effective amount of prochlorperazine (PCP), or analog thereof, in combination of an administration of a chemotherapeutic drug if the specific marker is overexpressed on cancer cells or their extracellular vesicles (EVs);
wherein the specific marker is selected from the group consisting of ERBB family member, CD9, and integrins.

2. The method of claim 1, wherein the ERBB family member is selected from the group consisting of HER1 (epidermal growth factor receptor (EGFR)), HER2, and HER3.

3. The method of claim 1, wherein the integrins is selected from the group consisting of integrin α6 subunit and integrin β4 subunit.

4. The method of claim 1, wherein the specific marker is selected from the group consisting of EGFR, p-EGFR, Glypican 1 (GPC1), EpCAM, CD9, integrins, α-Enolase (ENO1), HER2, and MET.

5. The method of claim 1, wherein the cancer is a chemotherapeutic drug-resistant cancer.

6. The method of claim 1, wherein the cancer is a metastatic cancer.

7. The method of claim 1, wherein the chemotherapeutic drug is an epidermal growth factor receptor (EGFR)-tyrosine kinase inhibitor.

8. The method of claim 7, wherein the EGFR-tyrosine kinase inhibitor is afatinib.

9. The method of claim 1, wherein the chemotherapeutic drug is gemcitabine.

10. The method of claim 1, wherein the analog of PCP is selected from the group consisting of trifluoperazine, fluphenazine, chlorpromazine, thioridazine, and perphenazine.

11. The method of claim 10, wherein the analog of PCP is thioridazine.

12. The method of claim 1, wherein the cancer is pancreatic cancer, head and neck cancer, or lung cancer.

13. The method of claim 12, wherein the pancreatic cancer is a pancreatic adenocarcinoma (PDAC).

14. The method of claim 12, wherein the head and neck cancer is head and neck squamous cell carcinoma (HNSCC).

15. The method of claim 12, wherein the lung cancer is non-small-cell lung carcinoma (NSCLC).

16. The method of claim 13, wherein the specific marker on pancreatic ductal adenocarcinoma (PDAC)-derived extracellular vesicles (EVs) is selected from the group consisting of EGFR, p-EGFR, Glypican 1 (GPC1), EpCAM, CD9, integrins, α-Enolase (ENO1), HER2, and MET.

17. A method for treating a cancer in a subject, which comprises administering to the subject a therapeutically effective amount of PCP or analog thereof; wherein the cancer is selected from the group consisting of pancreatic cancer, head and neck cancer, and lung cancer.

18. The method of claim 17, wherein the pancreatic cancer is a pancreatic adenocarcinoma (PDAC).

19. The method of claim 17, wherein the head and neck cancer is head and neck squamous cell carcinoma (HNSCC).

20. The method of claim 17, wherein the lung cancer is non-small-cell lung carcinoma (NSCLC).

21. The method of claim 17, wherein the analog of PCP is selected from the group consisting of trifluoperazine, fluphenazine, chlorpromazine, thioridazine, and perphenazine.

22. The method of claim 17, wherein the analog of PCP is thioridazine.

23. The method of claim 17, which is in combination of an administration of a chemotherapeutic drug.

24. The method of claim 23, wherein the chemotherapeutic drug is an epidermal growth factor receptor (EGFR)-tyrosine kinase inhibitor.

25. The method of claim 23, wherein the EGFR-tyrosine kinase inhibitor is afatinib.

26. The method of claim 23, wherein the chemotherapeutic drug is gemcitabine.

27. An in vitro method for diagnosing pancreatic adenocarcinoma (PDAC) in a subject comprising the steps of
(a) collecting cancer cells or extracellular vesicles (EVs) of the subject;
(b) detecting the expression of a specific surface marker on the cancer cells or EVs collected in step (a); and
(c) diagnosing the subject as suffering from PDAC if the specific surface marked is expressed in step (b);
wherein the specific surface marker is selected from the group consisting of EGFR, Glypican 1 (GPC1), EpCAM, CD9, integrins, α-Enolase (ENO1), HER2, HER3, MET and combination thereof.

28. An in vitro method for diagnosing head and neck squamous cell carcinoma (HNSCC) in a subject comprising the steps of
(a) collecting cancer cells or extracellular vesicles (EVs) of the subject,
(b) detecting the expression of a specific surface marker on the cancer cells or EVs collected in step (a); and
(c) diagnosing the subject as suffering from HNSCC if the specific surface marked is expressed in step (b);
wherein the specific surface marker is selected from the group consisting of EGFR, HER2, HER3, MET and combination thereof.

29. An in vitro method for diagnosing non-small-cell lung carcinoma (NSCLC) in a subject comprising the steps of
(a) collecting cancer cells or extracellular vesicles (EVs) of the subject;
(b) detecting the expression of a specific surface marker on the cancer cells or EVs collected in step (a); and
(c) diagnosing the subject as suffering from NSCLC if the specific surface marked is expressed in step (b);
wherein the specific surface marker is selected from the group consisting of EGFR, HER3 and combination thereof.
